(19) United States
(12) Patent Application Publication
(10) Pub. No.: US 2021/0380942 A1
(43) Pub. Date: Dec. 9, 2021

(54) METHODS AND COMPOSITIONS FOR THE TREATMENT OF CANCER AND INFECTIOUS DISEASES

(71) Applicant: Jounce Therapeutics, Inc., Cambridge, MA (US)

(72) Inventors: Christopher HARVEY, Boston, MA (US); Deborah LAW, Hillsborough, CA (US); Amanda Elizabeth HANSON, Newton, MA (US); Martin Yu FAN, Boston, MA (US)

(21) Appl. No.: 17/288,376

(22) PCT Filed: Oct. 24, 2019
(86) PCT No.: PCT/US2019/057789

§ 371 Date: Apr. 23, 2021

Related U.S. Application Data
(60) Provisional application No. 62/750,030, filed on Oct. 24, 2018.

(51) Int. Cl. C12N 5/0783; A61K 35/17

(52) U.S. Cl. CPC C12N 5/0636; A61K 35/17; C12N 2501/2304; C12N 2501/2302; C12N 2501/2312; C12N 2501/06

(57) ABSTRACT

The invention provides compositions and methods for the treatment of cancer and infectious diseases.

### FIELD OF THE INVENTION

The invention relates to methods of treating cancer and methods for selecting treatment approaches for cancer.

### SEQUENCE LISTING

The instant application contains a Sequence Listing which has been submitted electronically in ASCII format and is hereby incorporated by reference in its entirety. Said ASCII copy, created on Oct. 23, 2019 is named “51266-008WO2_Sequence_Listing_10.23.2019_ST25” and is 33,243 bytes in size.

### BACKGROUND

ICOS (Inducible T-cell COStimulator; CD278) is a member of the B7/CD28/CTLA-4 immunoglobulin superfamily and is specifically expressed on T cells. Unlike CD28, which is constitutively expressed on T cells and provides co-stimulatory signals necessary for full activation of resting T cells, ICOS is expressed only after initial T cell activation.

ICOS has been implicated in diverse aspects of T cell responses (reviewed in Simpson et al., Curr. Opin. Immunol. 22: 326-332, 2010). It plays a role in the formation of germinal centers, T/B cell collaboration, and immunoglobulin class switching. ICOS-deficient mice show impaired germinal center formation and have decreased production of interleukin IL-10. These defects have been specifically linked to deficiencies in T follicular helper cells. ICOS also plays a role in the development and function of other T cell subsets, including Th1, Th2, and Th17. Notably, ICOS co-stimulates T cell proliferation and cytokine secretion associated with both Th1 and Th2 cells. Accordingly, ICOS knock-out mice demonstrate impaired development of autoimmune phenotypes in a variety of disease models, including diabetes (Th1), airway inflammation (Th2), and EAE neuro-inflammatory models (Th17).

In addition to its role in modulating T effector (Teff) cell function, ICOS also modulates T regulatory cells (Tregs). ICOS is expressed at high levels on Tregs, and has been implicated in Treg homeostasis and function.

Upon activation, ICOS, a disulfide-linked homodimer, induces a signal through the PI3K and AKT pathways. Subsequent signaling events result in expression of lineage specific transcription factors (e.g., T-bet, GATA-3) and, in turn, effects on T cell proliferation and survival.

ICOS ligand (ICOSL; B7-H2; B7RP1; CD275; GL50), also a member of the B7 superfamily, is the only ligand for ICOS and is expressed on the cell surfaces of B cells, macrophages, and dendritic cells. ICOSL functions as a non-covalently linked homodimer on the cell surface in its interaction with ICOS. Human ICOSL, although not mouse ICOSL, has been reported to bind to human CD28 and CTLA-4 (Yao et al., Immunity 34: 729-740, 2011).

### SUMMARY

The invention provides methods of generating populations ICOShi, CD4+ T cells, the methods including isolating immune cells (e.g., peripheral blood mononuclear cells) from a patient, contacting the immune cells with (i) one or more antigens, and (ii) an ICOS agonist, wherein the contacting results in the generation of a population of ICOShi CD4+ T-cells.

In some embodiments, the contacting with one or more antigen precedes contacting with the ICOS agonist.

In some embodiments, the contacting of the immune cells with the antigen further includes contacting the immune cells with one or more Th1 skewing compounds and anti-CD28.

In some embodiments, the one or more Th1 skewing compounds are selected from the group consisting of IL-12, IL-2, anti-IL-4, IL-15, IL-18, and IL-21 (e.g., the one or more Th1 skewing compounds include IL-12, IL-2, and anti-IL-4).

In some embodiments, the ICOS agonist is an anti-ICOS antibody agonist, e.g., wherein the anti-ICOS antibody agonist includes (a) a heavy chain including the amino acid sequence of SEQ ID NO: 1 and/or (b) a light chain including the amino acid sequence of SEQ ID NO: 2.

In some embodiments, the anti-ICOS antibody agonist is selected from the group consisting of JTX-2011, BMS-986226, and GSK3359609.

In some embodiments, the methods include contacting the immune cells with two or more antigens (e.g., 10 or more antigens, 100 or more antigens, 1000 or more antigens, or 10,000 or more antigens).

In some embodiments, one or more of the antigens is an antigen associated with an infectious disease (e.g., HIV, tuberculosis, Epstein Barr virus, herpes simplex virus, malaria, and human papilloma virus).

In some embodiments, one or more of the antigens is a cancer-associated antigen.

In some embodiments, one or more of the cancer-antigens results from a mutation in a gene selected from the group consisting of: TP53, PIK3CA, TTN, KRAS, APC, MUC16, KMT2D, KMT2C, ARID1A, PTEN, BRAF, IDH1, NRAS, AKT1, and EGFR.

In some embodiments, one or more of the cancer-antigens results from a mutation selected from the group consisting of: TP53 (R273, R248, R175, R213, G245, R282, Y220, and H179), PIK3CA (H1047, E545, and E542), TTN, KRAS (G12, G13, and 061), APC, MUC16, KMT2D, KMT2C, ARID1A, PTEN (R130), BRAF (V600), IDH1 (R132), NRAS (Q61), AKT1 (E17), and EGFR (745-759 in-frame indel).

In some embodiments, one or more of the cancer-antigen results from a mutation identified in Chang et al., Cancer Discovery 8.2 (2018): 174-183 or Chang et al., Nature Biotechnology 34.2 (2016): 155.

In some embodiments, contacting the immune cells with an ICOS agonist further includes contacting the immune cells with an additional immunomodulatory agent.

In some embodiments, the additional immunomodulatory agent is selected from the group consisting of an anti-CD3 antibody, an anti-PD-1 antagonist antibody, an anti-PD-L1 antagonist antibody, an anti-CTLA4 antibody.

In some embodiments, the anti-PD-1 or anti-PD-L1 antagonist antibody is selected from the group consisting of avelumab, atezolizumab, CX-072, pembrolizumab, nivolumab, cemiplimab, spartalizumab, tislelizumab, JNJ-63723283, genolimzumab, AMP-514, AGEN2034, durvalumab, and JNC-1.

In some embodiments, the anti-CTLA4 antibody is selected from the group consisting of ipilimumab, tremelimumab, and BMS-986249.

In some embodiments, the method further includes isolating the ICOShi, CD4+ T cells.

In some embodiments, the method further includes culturing the ICOShi, CD4+ T cells.

In some embodiments, the method further includes culturing the isolated ICOShi, CD4+ T cells.

In some embodiments, the culturing includes initial culture conditions suitable for expanding the population of ICOShi, CD4+ T cells.

In some embodiments, the initial conditions suitable for expanding the population ICOShi, CD4+ T cells includes contacting the suspension of CD4+ T cells with a CD3 agonist such as, for example, an anti-CD3 antibody (e.g., OKT3).

In some embodiments, the initial conditions suitable for expanding the population of ICOShi, CD4+ T cells include contacting the cells with one or more of an anti-PD-1 antibody antagonist, an anti-CTLA-4 antibody, and an ICOS agonist.

In some embodiments, the initial conditions suitable for expanding the population of ICOShi, CD4+ T cells include contacting the cells with one or more compounds (e.g., two or more, or all three) selected from the group consisting of IL-2, IL-12 and anti-IL-4.

In some embodiments, the initial conditions suitable for expanding the population of ICOShi, CD4+ T cells include contacting the cells with an anti-CD28 antibody agonist.

In some embodiments, the CD3 agonist and anti-CD28 agonist are present in a tetrameric antibody complex.

In some embodiments, the suspension of ICOShi, CD4+ T cells are incubated under the initial culture conditions for a period between one and five days (e.g., approximately 1, 2, 3, 4, or five days).

In some embodiments, the method further includes incubating the suspension of ICOShi, CD4+ T cells under a second culture condition suitable for expanding the population of ICOShi, CD4+ T cells.

In some embodiments, the cells are washed prior to the application of the second culture condition.

In some embodiments, the second culture condition includes contacting the cells with one or more of an anti-PD-1 antibody antagonist, an anti-CTLA-4 antibody, and an ICOS agonist.

In some embodiments, the second culture condition includes contacting the cells with one or more compounds (e.g., two or more, or all three) selected from the group consisting of IL-2, IL-12 and anti-IL-4.

In some embodiments, the second culture condition includes contacting the cells with an anti-CD28 antibody agonist.

In some embodiments, the second culture conditions does not include contacting the cells with a CD3 agonist and/or CD28 agonist.

In some embodiments, the second culture condition is maintained for between 1 and 5 days (e.g., for 1, 2, 3, 4, or 5 days).

The invention also includes suspensions of cells generated by any one of the methods described herein, as well as pharmaceutical compositions including such cells (and suspensions thereof).

In some embodiments, the methods described above further include isolating the ICOShi, CD4+ T cells after the culturing.

In some embodiments, the isolating includes contacting the cells with an anti-ICOS antibody that does not compete with binding to ICOS with the ICOS agonist.

In some embodiments, the isolating includes contacting the cells with an antibody specific for the ICOS agonist (e.g., an anti-human-IgG1 antibody), or with protein A/G beads.

In some embodiments, the methods further include administering the ICOShi CD4+ T-cells (e.g., the isolated ICOShi CD4+ T-cells) to the patient, thereby treating the patient for a disease associated with the one or more antigens.

In some embodiments, the one or more antigens is associated with cancer and the disease is cancer.

In some embodiments, the cancer is selected from gastric cancer, breast cancer, which optionally is triple negative breast cancer (TNBC), non-small cell lung cancer (NSCLC), melanoma, renal cell carcinoma (RCC), bladder cancer, endometrial cancer, diffuse large B-cell lymphoma (DLBCL), Hodgkin's lymphoma, ovarian cancer, and head and neck squamous cell cancer (HNSCC).

In some embodiments, the one or more antigens is associated with an infectious disease and the disease is an infectious disease.

In some embodiments, prior to the isolation of the immune cells from the patient, the patient is treated with an anti-cancer treatment.

In some embodiments, the anti-cancer treatment is selected from the group consisting of an agonist to TLR9, TLR2/4, TLR7/8, and/or TLR7, radiation, chemotherapies, tyrosine kinase inhibitors, epigenetic modifiers (e.g., HDAC inhibitors and demethylating agents), IMIDs (immuno-modulatory drugs, e.g., lenalidomide), cytokines (e.g., interferon gamma), anti-CTLA-4 antibodies, oncolytic viruses, vaccines, CD40 agonists, and ICOS agonists.

In some embodiments, the anti-CTLA-4 antibody is selected from the group consisting of ipilimumab, tremelimumab, and BMS-986249.

In some embodiments, the anti-cancer treatment ICOS agonist is an anti-ICOS antibody agonist, e.g., wherein the anti-ICOS antibody agonist includes (a) a heavy chain including the amino acid sequence of SEQ ID NO: 1 and/or (b) a light chain including the amino acid sequence of SEQ ID NO: 2.

In some embodiments, the anti-cancer treatment anti-ICOS antibody agonist is selected from the group consisting of JTX-2011, BMS-986226, and GSK3359609.

The invention also provides populations of ICOShi, CD4+ T-cells generated by the methods described above and elsewhere herein.

In some embodiments of the methods described above and elsewhere herein, the patient is determined to have a disease associated with the one or more antigens prior to the contacting of the immune cells with the one or more antigens.

The invention also provides methods of treating a subject having cancer or an infectious disease, the methods including administering to the subject ICOShi CD4+ T-cells, wherein the cells are optionally obtained using a method described above or elsewhere herein.

The invention further provides methods for generating a population of ICOShi CD4+ T cells, the method including contacting immune cells isolated from a patient with (i) one or more antigens, and (ii) an ICOS agonist, wherein the contacting results in the generation of a population of ICOShi CD4+ T-cells. Additional details of this method can optionally be as described above or elsewhere herein.

The invention additionally includes the use of one or more antigens and an ICOS agonist for the generation of a population of ICOShi CD4+ T cells from immune cells isolated from a patient, e.g., as described above or elsewhere herein.

Furthermore, the invention provides the use of ICOShi CD4+ T-cells in the treatment of a subject having cancer or an infectious disease, wherein the cells are optionally obtained using a method described above or elsewhere herein.

In some embodiments of any of the methods described above and elsewhere herein, the subject has an infectious disease (e.g., HIV, tuberculosis, Epstein Barr virus, herpes simplex virus, malaria, or human papilloma virus) and the methods of the invention are optionally used to treat the infectious disease.

Other features and advantages of the invention will be apparent from the following detailed description, the drawings, and the claims.

### BRIEF DESCRIPTION OF THE DRAWINGS

FIG. 1 is a series of histograms showing magnitude of expression of ICOS as a function of concentration of OKT-3 in the media. Three donors are shown.

FIG. 2 is a series of histograms indicating cytokine expression from the indicated cell population. Upper plots show cytokine production in isolated ICOS hi CD4+ T cells while lower plots show cytokine production in ICOS low cells. In each plot, the left set of histograms were obtained from culture conditions absent JTX-2011 re-stimulation while the right set of histograms were obtained from culture conditions where JTX-2011 re-stimulation was present. Cytokine levels are measured through conjugation to fluorescent probes of differing wave-lengths. Y-axis shows mean fluorescent intensity level associated with the indicated cytokine. Experiments based on PBMCs isolated from three donors are shown.

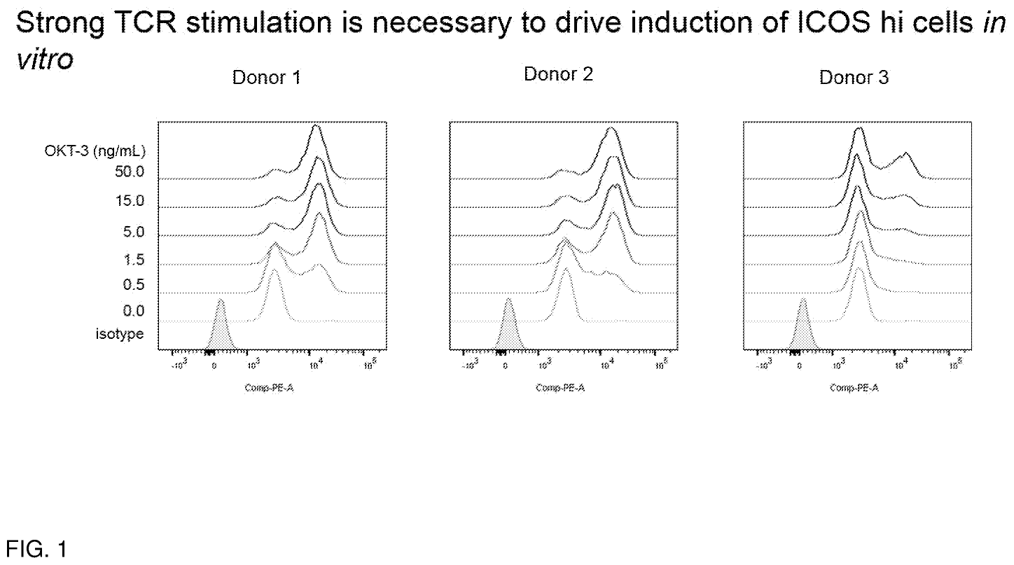

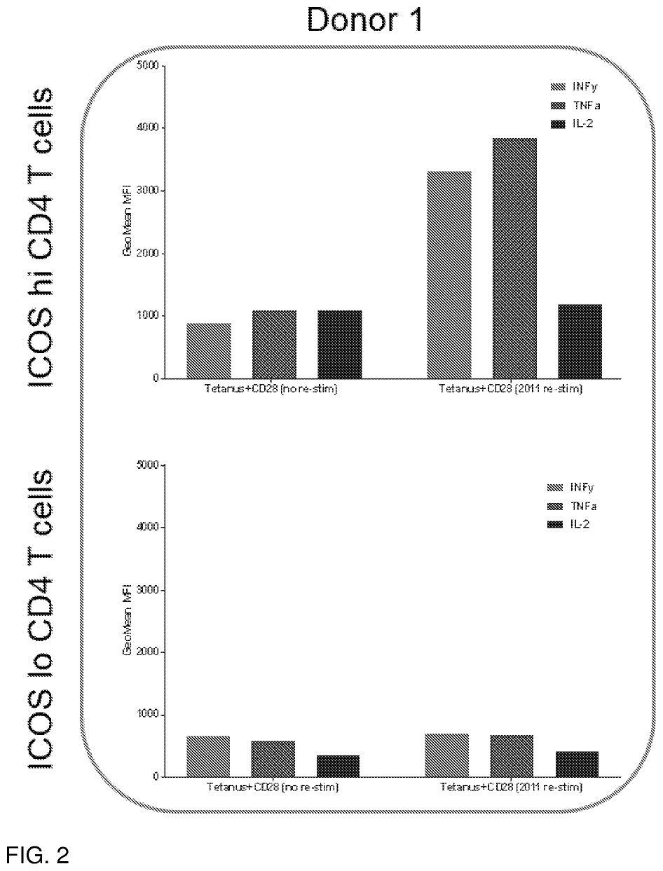

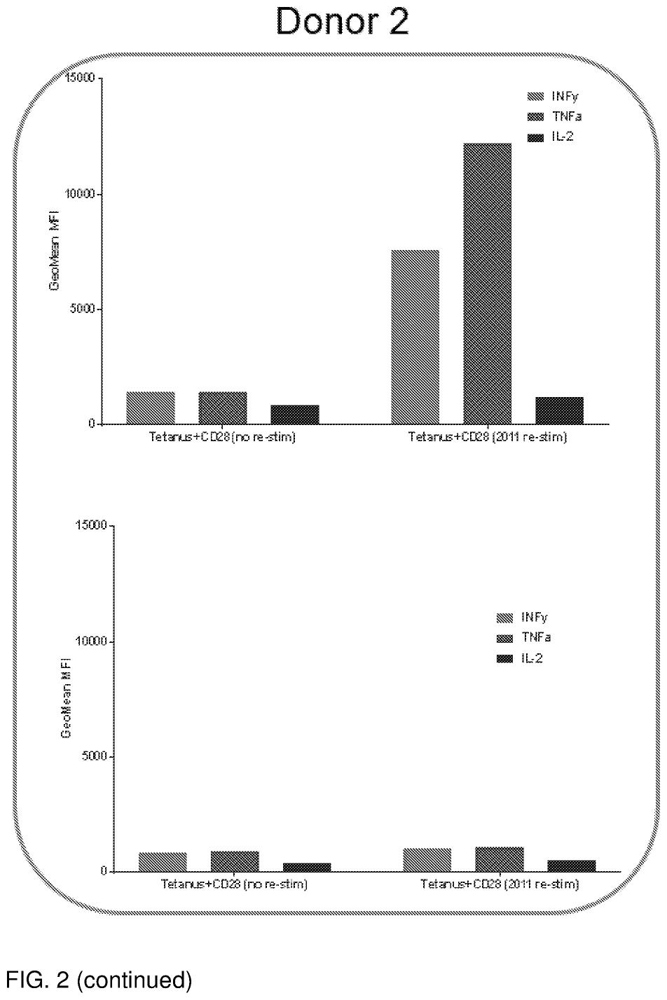

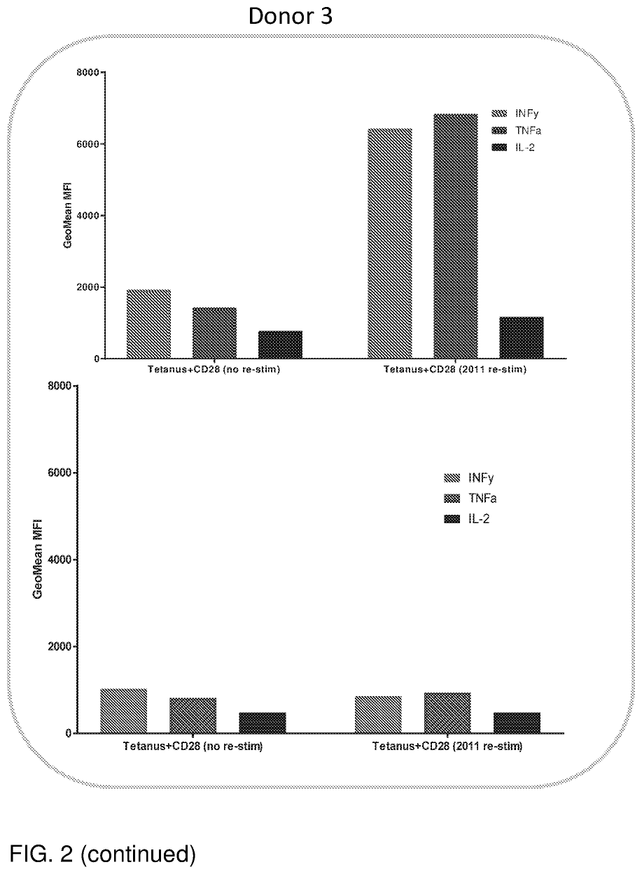

### DETAILED DESCRIPTION

Methods of generating populations of CD4+ T cells having elevated levels of ICOS (i.e., ICOS^{high} or ICOS^{hi} CD4+ T cells) are provided. In general, these methods include contacting immune cells obtained from a subject (e.g., isolated peripheral blood mononuclear cells; PBMCs) with one or more antigens and an ICOS agonist. The one or more antigens can optionally be characteristic of a disease including, e.g., cancer or an infectious disease. ICOS^{high} CD4+ T cells, such as those generated using the methods of the invention, can be used to prevent, improve, or treat diseases by administration to a subject (e.g., the subject from whom the immune cells were originally obtained). The invention also provides ICOS^{high} CD4+ T cells, such as those made using the methods of the invention, as well as pharmaceutical compositions including such cells. The methods and compositions of the invention are described further as follows.

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

### I. Definitions

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments of the invention described herein include “consisting” and/or “consisting essentially of” embodiments.

As used herein, the singular form “a,” “an,” and “the” includes plural references unless indicated otherwise.

Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

As is understood by one skilled in the art, reference to “about” a value or parameter herein includes (and describes) embodiments that are directed to that value or parameter per se. For example, description referring to “about X” includes description of “X.”

The terms “nucleic acid molecule,” “nucleic acid,” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

“ICOS” and “inducible T-cell costimulatory” as used herein refer to any native ICOS that results from expression and processing of ICOS in a cell. The term includes ICOS from any vertebrate source, including mammals such as primates (e.g., humans and cynomolgus monkeys) and rodents (e.g., mice and rats), unless otherwise indicated. The term also includes naturally occurring variants of ICOS, e.g., splice variants or allelic variants. The amino acid sequence of an exemplary human ICOS precursor protein, with signal sequence (amino acids 1-20) is shown in SEQ ID NO: 11. The amino acid sequence of an exemplary mature human ICOS is shown in SEQ ID NO: 12. The intracellular portion of ICOS is indicated in Table 3 by underlining within SEQ ID NOs: 11 and 12. The amino acid sequence of an exemplary mouse ICOS precursor protein, with signal sequence (amino acids 1-20) is shown in SEQ ID NO: 13. The amino acid sequence of an exemplary mature mouse ICOS is shown in SEQ ID NO: 14. The amino acid sequence of an exemplary rat ICOS precursor protein, with signal sequence (amino acids 1-20) is shown in SEQ ID NO: 15. The amino acid sequence of an exemplary mature rat ICOS is shown in SEQ ID NO: 16. The amino acid sequence of an exemplary cynomolgus monkey ICOS precursor protein, with signal sequence (amino acids 1-20) is shown in SEQ ID NO: 17. The amino acid sequence of an exemplary mature cynomolgus monkey ICOS is shown in SEQ ID NO: 18.

The term “specifically binds” to an antigen or epitope is a term that is well-understood in the art, and methods to determine such specific binding are also well known in the art. A molecule is said to exhibit “specific binding” or “preferential binding” if it reacts or associates more frequently, more rapidly, with greater duration, and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an ICOS epitope is an antibody that binds this epitope with greater affinity, avidity, more readily, and/or with greater duration than it binds to other ICOS epitopes or non-ICOS epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, “substantially pure” refers to material which is at least 50% pure (that is, free from contaminants), more preferably, at least 90% pure, more preferably, at least 95% pure, yet more preferably, at least 98% pure, and most preferably, at least 99/o pure.

As used herein, the term “epitope” refers to a site on a target molecule (for example, an antigen, such as a protein, nucleic acid, carbohydrate, or lipid) to which an antigen-binding molecule (for example, an antibody, antibody fragment, or scaffold protein containing antibody binding regions) binds. Epitopes often include a chemically active surface grouping of molecules such as amino acids, polypeptides, or sugar side chains and have specific three-dimensional structural characteristics as well as specific charge characteristics. Epitopes can be formed both from contiguous and/or juxtaposed noncontiguous residues (for example, amino acids, nucleotides, sugars, or lipid moieties) of the target molecule. Epitopes formed from contiguous residues, also called linear epitopes (for example, amino acids, nucleotides, sugars, or lipid moieties), typically are retained on exposure to denaturing solvents whereas epitopes formed from non-contiguous residues, also called non-linear or conformational epitopes, are formed by tertiary folding, and typically are lost on treatment with denaturing solvents. An epitope may include, but is not limited to, at least 3, at least 5, or 8-10 residues (for example, amino acids or nucleotides). In some examples, an epitope is less than 20 residues (for example, amino acids or nucleotides) in length, less than 15 residues, or less than 12 residues.

Two antibodies may bind to the same epitope within an antigen, or to overlapping epitopes, if they exhibit competitive binding for the antigen. Accordingly, in some embodiments, an antibody is said to “cross-compete” with another antibody if it specifically interferes with the binding of the antibody to the same or an overlapping epitope.

The term “antibody” herein is used in the broadest sense and encompasses various antibody structures, including but not limited to, monoclonal antibodies, polyclonal antibodies, multispecific antibodies (for example, bispecific (such as Bi-specific T-cell engagers) and trispecific antibodies), and antibody fragments as long as they exhibit a desired antigen-binding activity.

The term antibody includes, but is not limited to, fragments that are capable of binding to an antigen, such as Fv, single-chain Fv (scFv), Fab, Fab′, di-scFv, sdAb (single domain antibody), and (Fab′)2 (including a chemically linked F(ab′)2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc. Furthermore, for all antibody constructs provided herein, variants having the sequences from other organisms are also contemplated. Thus, if a human version of an antibody is disclosed, one of skill in the art will appreciate how to transform the human sequence based antibody into a mouse, rat, cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, and/or a combination of the Kabat, Chothia, AbM, and/or contact definitions. Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991)). The AbM definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, H26-H35B of H1, 50-58 of H2, and 95-102 of H3. The Contact definition can include, for example, CDRs (CDR-L1, CDR-L2, CDR-L3, CDR-H1, CDR-H2, and CDR-H3) at amino acid residues 30-36 of L1, 46-55 of L2, 89-96 of L3, 30-35 of H1, 47-58 of H2, and 93-101 of H3. The Chothia definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, 26-32 . . . 34 of H1, 52-56 of H2, and 95-102 of H3. With the exception of CDR1 in V_{H}, CDRs generally comprise the amino acid residues that form the hypervariable loops. The various CDRs within an antibody can be designated by their appropriate number and chain type, including, without limitation as: a) CDR-L1, CDR-L2, CDR-L3, CDR-H1, CDR-H2, and CDR-H3; b) CDRL1, CDRL2, CDRL3, CDRH1, CDRH2, and CDRH3; c) LCDR-1, LCDR-2, LCDR-3, HCDR-1, HCDR-2, and HCDR-3; or d) LCDR1, LCDR2, LCDR3, HCDR1, HCDR2, and HCDR3; etc. The term “CDR” is used herein to also encompass HVR or a “hyper variable region,” including hypervariable loops. Exemplary hypervariable loops occur at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)).

The term “heavy chain variable region” as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “heavy chain constant region” as used herein refers to a region comprising at least three heavy chain constant domains, CH1, CH2, and CH3. Of course, non-function-altering deletions and alterations within the domains are encompassed within the scope of the term “heavy chain constant region,” unless designated otherwise. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an E constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising at least three light chain CDRs. In some embodiments, the light chain variable region includes the three CDRs and at least FR2 and FR3. In some embodiments, the light chain variable region includes at least light chain LCR1, framework (FR) 2, LCD2, FR3, and LCD3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “light chain constant region” as used herein refers to a region comprising a light chain constant domain, C_{L}. Non-limiting exemplary light chain constant regions include λ and K. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “acceptor human framework” for the purposes herein is a framework comprising the amino acid sequence of a light chain variable domain (V_{L}) framework or a heavy chain variable domain (V_{H}) framework derived from a human immunoglobulin framework or a human consensus framework, as defined below. An acceptor human framework derived from a human immunoglobulin framework or a human consensus framework can comprise the same amino acid sequence thereof, or it can contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L} acceptor human framework is identical in sequence to the V_{L} human immunoglobulin framework sequence or human consensus framework sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the dissociation constant (KD). Affinity can be measured by common methods known in the art (such as, for example, ELISA KD, KinExA, bio-layer interferometry (BLI), and/or surface plasmon resonance devices (such as a BIAcore® device), including those described herein).

The term “KD,” “Kd,” “Kd,” or “Kd value” as used herein, refers to the equilibrium dissociation constant of an antibody-antigen interaction.

The term “biological activity” refers to any one or more biological properties of a molecule (whether present naturally as found in vivo, or provided or enabled by recombinant means). Biological properties include, but are not limited to, binding a receptor, inducing cell proliferation, inhibiting cell growth, inducing other cytokines, inducing apoptosis, and enzymatic activity. In some embodiments, biological activity of an ICOS protein includes, for example, costimulation of T cell proliferation and cytokine secretion associated with Th1 and Th2 cells; modulation of Treg cells; effects on T cell differentiation including modulation of transcription factor gene expression; induction of signaling through PI3K and AKT pathways; and mediating ADCC.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

The phrase “substantially different,” as used herein, denotes a sufficiently high degree of difference between two numeric values such that one of skill in the art would consider the difference between the two values to be of statistical significance within the context of the biological characteristic measured by said values. In some embodiments, the two substantially different numeric values differ by greater than about any one of 10%, 20%, 25%, 30%, 35%, 40%, 45%, 50%, 60%, 70%, 80%, 90%, or 100%.

The phrase “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 20%, 25%, 30%, 35%, 40%, 45%, 50%, 60%, 70%, 80%, 90%, or 100% compared to the reference value.

The phrase “substantially increased,” as used herein, denotes a sufficiently high degree of increase between a numeric value and a reference numeric value such that one of skill in the art would consider the difference between the two values to be of statistical significance within the context of the biological characteristic measured by said values. In some embodiments, the substantially increased numeric values is increased by greater than about any one of 10%, 20%, 25%, 30%, 35%, 40%, 45%, 50%, 60%, 70%, 80%, 90%, or 100% compared to the reference value.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.”

The terms “individual,” “patient,” or “subject” are used interchangeably herein to refer to an animal, for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

The term “sample” or “patient sample” as used herein, refers to a composition that is obtained or derived from a subject of interest that contains a cellular and/or other molecular entity that is to be characterized and/or identified, for example, based on physical, biochemical, chemical, and/or physiological characteristics. For example, the phrase “test sample,” and variations thereof, refers to any sample obtained from a subject of interest that would be expected or is known to contain a cellular and/or molecular entity that is to be characterized. By “tissue or cell sample” is meant a collection of similar cells obtained from a tissue of a subject or patient. The source of the tissue or cell sample may be blood (e.g., peripheral blood) or any blood constituents; solid tissue as from a fresh, frozen, and/or preserved organ or tissue sample or biopsy or aspirate; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like. In some embodiments, a sample includes peripheral blood obtained from a subject or patient, which includes CD4+ cells. In some embodiments, a sample includes CD4+ cells isolated from peripheral blood. In some embodiments, a sample is a sample of peripheral blood mononuclear cells (PBMCs).

A “control,” “control sample,” “reference,” or “reference sample” as used herein, refers to any sample, standard, or level that is used for comparison purposes. A control or reference may be obtained from a healthy and/or non-diseased sample. In some examples, a control or reference may be obtained from an untreated sample or patient. In some examples, a reference is obtained from a non-diseased or non-treated sample of a subject individual. In some examples, a reference is obtained from one or more healthy individuals who are not the subject or patient. In some embodiments, a control sample, reference sample, reference cell, or reference tissue is obtained from the patient or subject at a time point prior to one or more administrations of a treatment (e.g., one or more anti-cancer treatments), or prior to being subjected to any of the methods of the invention.

A “disease” or “disorder” as used herein refers to a condition where treatment is needed and/or desired. In some embodiments, the disease or disorder is cancer.

“Cancer” and “tumor,” as used herein, are interchangeable terms that refer to any abnormal cell or tissue growth or proliferation in an animal. As used herein, the terms “cancer” and “tumor” encompass solid and hematological/lymphatic cancers and also encompass malignant, pre-malignant, and benign growth, such as dysplasia. Examples of cancer include but are not limited to, carcinoma, lymphoma, blastoma, sarcoma, and leukemia. More particular non-limiting examples of such cancers include gastric cancer, breast cancer (e.g., triple negative breast cancer (TNBC)), non-small cell lung cancer (NSCLC), squamous cell cancer, small-cell lung cancer, pituitary cancer, esophageal cancer, astrocytoma, soft tissue sarcoma, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, colon cancer, colorectal cancer, endometrial or uterine carcinoma (including uterine corpus endometrial carcinoma), salivary gland carcinoma, kidney cancer, renal cancer, liver cancer, prostate cancer, vulval cancer, thyroid cancer, hepatic carcinoma, brain cancer, testis cancer, cholangiocarcinoma, gallbladder carcinoma, melanoma, and various types of head and neck cancer. These cancers, and others, can be treated or analyzed according to the methods of the invention.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example, metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering an anti-cancer therapy. “Ameliorating” also includes shortening or reduction in duration of a symptom.

In the context of cancer, the term “treating” includes any or all of: inhibiting growth of cancer cells, inhibiting replication of cancer cells, lessening of overall tumor burden, and ameliorating one or more symptoms associated with the disease.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce,” “inhibit,” or “prevent” do not denote or require complete prevention over all time.

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (for example, severity of disease, progression/non-progression/improvement, etc.). While the present disclosure may provide exemplary predetermined levels, it is well-known that cutoff values may vary depending on the nature of the immunoassay (for example, antibodies employed, etc.). It further is well within the skill of one of ordinary skill in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) may be generally applicable.

In some embodiments, the terms “elevated levels of ICOS,” “elevated ICOS levels,” “ICOS at an elevated level,” “ICOS^{HIGH},” and “ICOS^{hi}” refer to increased levels of ICOS in cells (e.g., CD4+ T cells) of a subject, e.g., in a peripheral blood sample of the subject, after treatment of the subject with one or more anti-cancer therapies. The increased levels can be determined relative to a control which may be, e.g., a peripheral blood sample from the subject being treated, but either before any treatment with the one or more anti-cancer therapies at all, or before treatment with a second or further cycle of the one or more anti-cancer therapies. Alternatively, the control can be a level from a matched sample (e.g., a peripheral blood sample) of a healthy individual. In some embodiments, the level of ICOS is determined at the level of expressed protein, which may be detected in some embodiments using an antibody directed to an intracellular portion of ICOS. In some embodiments, the detection using such an antibody is done by use of flow cytometry. In some embodiments, an increase of at least 2-fold (e.g., at least 3-fold, 4-fold, 5-fold, 7.5-fold, 10-fold, or 15-fold) in mean fluorescence intensity (MFI), relative to a control, indicates detection of elevated ICOS levels. In some embodiments, detection of an increase in ICOS levels in at least 5% (e.g., at least 10%, 20%, 30%, 40%, 50%, 60%, 70%, 80%, or 90%) of CD4+ T cells in a peripheral blood sample indicates a subject having an ICOS hi sample. In some embodiments, an increase of at least 2-fold (e.g., at least 3-fold, 4-fold, 5-fold, 7.5-fold, 10-fold, or 15-fold) in mean fluorescence intensity (MFI), relative to a control, in at least 5% (e.g., at least 10%, 20%, 30%, 40%, 50%, 60%, 70%, 80%, or 90%) of CD4+ T cells in a peripheral blood sample indicates detection of elevated ICOS levels. In some embodiments, elevated ICOS levels refer to an increase in total ICOS expression levels (e.g., mRNA levels or protein levels) in CD4+ T cells in the peripheral blood test sample of about 10%, 20%, 30%, 40%, 50%, 60%, 70%, 80%, 85%, 90%, 95%, 100%, or greater relative to a control sample. In some embodiments, elevated ICOS levels refers to an increase in total ICOS expression levels (e.g., mRNA levels or protein levels) in the CD4+ T cells in a peripheral blood sample of about at least 1.1×, 2×, 3×, 4×, 5×, 10×, 15×, 20×, 30×, 40×, 50×, 100×, 500×, 1000×, or greater relative to a control sample.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce, or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time.

As used herein, “delaying development of a disease” means to defer, hinder, slow, retard, stabilize, suppress, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

A “therapeutically effective amount” of a substance/molecule, agonist, or antagonist may vary according to factors such as the disease state, age, sex, and weight of the individual, and the ability of the substance/molecule, agonist, or antagonist to elicit a desired response in the individual. A therapeutically effective amount is also one in which any toxic or detrimental effects of the substance/molecule, agonist, or antagonist are outweighed by the therapeutically beneficial effects. A therapeutically effective amount may be delivered in one or more administrations. A therapeutically effective amount refers to an amount effective, at dosages, and for periods of time necessary, to achieve the desired therapeutic and/or prophylactic result. The therapeutically effective amount of the treatment of the invention can be measured by various endpoints commonly used in evaluating cancer treatments, including, but not limited to: extending survival (including OS and PFS); resulting in an objective response (including a CR or a PR); tumor regression, tumor weight or size shrinkage, longer time to disease progression, increased duration of survival, longer PFS, improved OS rate, increased duration of response, and improved quality of life and/or improving signs or symptoms of cancer.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time, or where the administration of one therapeutic agent falls within a short period of time (e.g., within one day) relative to administration of the other therapeutic agent. For example, the two or more therapeutic agents are administered with a time separation of no more than about a specified number of minutes.

The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes.

As used herein, “in conjunction with” refers to administration of one treatment modality in addition to another treatment modality. As such, “in conjunction with” refers to administration of one treatment modality before, during, or after administration of the other treatment modality to the individual.

The terms “label” and “detectable label” mean a moiety attached to a polynucleotide or polypeptide to render a reaction (for example, polynucleotide amplification or antibody binding) detectable. The polynucleotide or polypeptide comprising the label may be referred to as “detectably labeled.” Thus, the term “labeled binding protein” refers to a protein with a label incorporated that provides for the identification of the binding protein. The term “labeled oligonucleotide,” “labeled primer,” “labeled probe,” etc. refers to a polynucleotide with a label incorporated that provides for the identification of nucleic acids that comprise or are hybridized to the labeled oligonucleotide, primer, or probe. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In some embodiments, the moiety itself may not be detectably labeled but may become detectable upon reaction with yet another moiety.

The term “conjugate” refers to an antibody that is chemically linked to a second chemical moiety, such as a therapeutic or cytotoxic agent. The term “agent” includes a chemical compound, a mixture of chemical compounds, a biological macromolecule, or an extract made from biological materials. In some embodiments, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody may be a detectably labeled antibody used as the detection antibody.

As used herein, the term “flow cytometry” generally refers to a technique for characterizing biological particles, such as whole cells or cellular constituents, by flow cytometry. Methods for performing flow cytometry on samples of immune cells are well known in the art (see e.g., Jaroszeski et al., Method in Molecular Biology (1998), vol. 91: Flow Cytometry Protocols, Humana Press; Longobanti Givan, (1992) Flow Cytometry, First Principles, Wiley Liss). All known forms of flow cytometry are intended to be included, particularly fluorescence activated cell sorting (FACS), in which fluorescent labeled molecules are evaluated by flow cytometry.

The term “amplification” refers to the process of producing one or more copies of a nucleic acid sequence or its complement. Amplification may be linear or exponential (e.g., PCR).

The technique of “polymerase chain reaction” or “PCR” as used herein generally refers to a procedure wherein a specific region of nucleic acid, such as RNA and/or DNA, is amplified as described, for example, in U.S. Pat. No. 4,683,195. Generally, oligonucleotide primers are designed to hybridize to opposite strands of the template to be amplified, a desired distance apart. PCR can be used to amplify specific RNA sequences, specific DNA sequences from total genomic DNA, and cDNA transcribed from total cellular RNA, bacteriophage or plasmid sequences, etc.

“Quantitative real time PCR” or “qRT-PCR” refers to a form of PCR wherein the PCR is performed such that the amounts, or relative amounts of the amplified product can be quantified. This technique has been described in various publications including Cronin et al., Am. J. Pathol. 164(1):35-42 (2004); and Ma et al., Cancer Cell 5:607-616 (2004).

The term “target sequence,” “target nucleic acid,” or “target nucleic acid sequence” refers generally to a polynucleotide sequence of interest, e.g., a polynucleotide sequence that is targeted for amplification using, for example, qRT-PCR.

The term “detection” includes any means of detecting, including direct and indirect detection.

### II. Methods for Isolating Patient Immune Cells

Immune cells for use in the invention can be obtained using methods that are known in the art. In some embodiments, the immune cells are comprised within a blood sample from a patient (e.g., a peripheral blood sample). In some embodiments, the immune cells are peripheral blood mononuclear cells (PBMCs) that are obtained from a whole blood sample of a patient. PBMCs can be obtained from whole blood using standard methods including, e.g., isolation through a density gradient separation using BD Vacutainer CPT Mononuclear Cell Preparation.

### III. Culture Conditions for Initial Treatment of Cells

Immune cells (e.g., isolated PBMCs) for use in the methods of the invention can be cultured in cell culture medium that optionally includes one or more Th1 skewing agent and/or anti-CD28. The Th1 skewing agents can be any one or more of IL-12, IL-2, and anti-IL-4. For example, the Th1 skewing agents can be all three of IL-12, IL-2, and anti-IL-4. Optionally, one or more of the following Th1 skewing agents can be used, in addition to one or more (e.g., all) of IL-12, IL-2, and anti-IL-4, or separately (either individually or in combination): IL-15, IL-18, and IL-21. During culture, the immune cells are, in general, first exposed to an antigen and then subsequently they are exposed to an ICOS agonist, as described further below.

### IV. Methods of Antigen Exposure

Immune cells (e.g., isolated PBMCs), such as immune cells cultured under the conditions described above, can be exposed to one or more antigens using standard methods. For example, the one or more antigens can be attached to beads that are contacted with the immune cells. In another example, the one or more antigens are bound to a plate to which the immune cells are contacted. In a further example, the one or more antigens are soluble and are contacted with the immune cells in solution by mixing.

In some embodiments, the one or more antigens and the immune cells are incubated with one another for a period of, e.g., 24-96, e.g., 48-72 hours, at 37° C. Optionally, the incubation medium includes a Th1 skewing agent (see above, including the optional combinations noted above) and/or anti-CD28. After the incubation, stimulus is removed and the cells are washed. The cells are then contacted with an ICOS agonist, as described below.

### V. Exemplary Antigens

As set forth above, according to the methods of the invention, immune cells (e.g., PBMCs) are contacted with one or more antigens and an ICOS agonist. The one or more antigens can optionally be characteristic of a disease or condition that is to be prevented, improved, or treated using the ICOS^{high} CD4+ T-cells of the invention including, e.g., cancer or infectious diseases. There are a very large number of such antigens known in the art, and any of them can be used in the invention. Accordingly, any examples provided herein are not to be considered as limiting of the invention.

Examples of cancer-related antigens that can be used in the invention include those in the cBioPortal database (Cerami et al., The cBio Cancer Genomics Portal: An Open Platform for Exploring Multidimensional Cancer Genomics Data. Cancer Discovery. May 2012 2; 401; Gao et al., Integrative Analysis of Complex Cancer Genomics and Clinical Profiles using the cBioPortal. Sci. Signal. 6, pI1 (2013)). This database and these references are incorporated herein by reference. The following list provides specific examples of cancer-related antigens from the cBioPortal database that can be used in the invention, with exemplary mutations in the antigens indicated parenthetically in some instances. The mutations may optionally each be present as single residue mutations, unless indicated otherwise. The list is as follows: TP53 (R273, R248, R175, R213, G245, R282, Y220, and H179), PIK3CA (H1047, E545, and E542), TTN, KRAS (G12, G13, and 061), APC, MUC16, KMT2D, KMT2C, ARID1A, PTEN (R130), BRAF (V600), IDH1 (R132), NRAS (061), AKT1 (E17), and EGFR (745-759 in-frame indel); (also see Chang et al., “Accelerating discovery of functional mutant alleles in cancer,” Cancer Discovery 8.2 (2018): 174-183; and Chang et al., “Identifying recurrent mutations in cancer reveals widespread lineage diversity and mutational specificity,” Nature Biotechnology 34.2 (2016): 155; each of which is incorporated herein by reference).

In certain aspects, the invention features first determining the presence of one or more disease-associated antigens in a patient, e.g., one or more cancer-associated antigens in a tumor of the patient. For example, a biopsy of the tumor could be sampled and sequenced to determine whether any particular antigens are present. Alternatively, cell free DNA or RNA can be sequenced, and candidate antigens can be identified. Accordingly, the invention encompasses both 1) methods where the isolated immune cells are exposed to a panel of pre-selected antigens even absent prior identification of the antigens in the patient, and 2) methods where the exposure of isolated immune cells antigens is designed to expose the immune cells to antigens (e.g., custom antigens) already determined to present in the patient's cancer cells.

Examples of infectious disease-related antigens include antigens from pathogens such as, for example, HIV, tuberculosis, Epstein Barr virus, herpes simplex virus, malaria, and human papilloma virus. Similar to the above, the invention in the context of infectious diseases can involve first determining the presence of one or more disease-associated antigens in a patient (e.g., by tissue biopsy or blood test). Alternatively, cell free DNA or RNA can be sequenced, and candidate antigens can be identified. Similar to the above, the invention encompasses in the infectious disease context both 1) methods where the isolated immune cells are exposed to a panel of pre-selected antigens even absent prior identification of the antigens in the patient, and 2) methods where the exposure of isolated immune cells antigens is designed to expose the immune cells to antigens (e.g., custom antigens) already determined to present in a patient.

The one or more antigens can optionally be full length antigen polypeptides, peptide fragments including effective antigenic determinants, or other molecules (e.g., carbohydrates). The number of antigens used is not limited and can be, e.g., 1-10, 10 or more, 100 or more, 1,000 or more, or 10,000 or more.

### VI. T Cell Stimulation and Expansion

T cells exposed to one or more antigens, as described above, are further exposed to one or more ICOS agonist for the generation of ICOS^{high} CD4+ T cells. As noted above, this is typically done after the T cells have been exposed to antigen. As non-limiting examples, the contacting with the one or more antigens can be done at least 1, 2, 4, 8, 10, 12, or 18 hours; 1, 2, 3, 4, 5, or 6 days; or 1, 2, 3, or 4 weeks before exposure to the one or more ICOS agonist. ICOS agonist treatment is optionally carried out in the presence of one or more Th1 skewing agents (see above, including the combinations noted above) and/or anti-CD28. Examples of ICOS agonists that can be used in this aspect of the invention are provided further below, and include JTX-2011, BMS-986226, and GSK3359609.

In addition to the one or more ICOS agonist, the T cells at this stage can also be contacted with one or more additional immunomodulatory agent including, e.g., an anti-CD3 antibody, an anti-PD-1 antagonist antibody, an anti-PD-L1 antagonist antibody, and/or an anti-CTLA4 antibody. In some embodiments, the anti-PD-1 or anti-PD-L1 antagonist antibody is selected from the group consisting of avelumab, atezolizumab, CX-072, pembrolizumab, nivolumab, cemiplimab, spartalizumab, tislelizumab, JNJ-63723283, genolimzumab, AMP-514, AGEN2034, durvalumab, and JNC-1. In some embodiments, the anti-CTLA4 antibody is selected from the group consisting of ipilimumab, tremelimumab, and BMS-986249.

ICOS^{high} CD4+ T cells obtained using, e.g., the methods described above can also be expanded for use in therapeutic methods. Accordingly, the invention also includes methods of generating expanded populations of CD4+ T cells having elevated ICOS expression. These methods including culturing ICOS^{high} CD4+ T cells, as described above, under initial culture conditions suitable for expanding the population of CD4+ T cells. These initial conditions may include contacting a suspension of the cells with, e.g., a CD3 agonist (e.g., OKT3), one or more of an anti-PD-1 antibody antagonist, an anti-CTLA-4 antibody, and an ICOS agonist, and, optionally, one or more compounds (e.g., two or more, or all three) selected from the group including of IL-2, IL-12, and anti-IL-4. These methods can also include contacting the suspension with, e.g., a CD28 agonist. In certain embodiments, the CD3 agonist and anti-CD28 agonist are present in a tetrameric antibody complex. In such methods, the suspension of CD4+ T cells are incubated under the initial culture conditions, e.g., for a period between one and five days (e.g., approximately 1, 2, 3, 4, or five days).

In certain embodiments, the methods of the invention can further include incubating the suspension of ICOS^{high} CD4+ T cells under a second culture condition suitable for expanding the population of cells. Here, optionally, the cells are washed prior to the application of the second culture conditions. In certain embodiments, the second culture condition can include, e.g., contacting the suspension of cells with an anti-PD-1 antibody antagonist, an anti-CTLA-4 antibody, and an ICOS agonist. In some embodiments, the second culture condition includes contacting the suspension of cells with one or more compounds (e.g., two or more, or all three) selected from the group including of IL-2, IL-12, and anti-IL-4. Additionally, or alternatively, the second culture condition includes contacting the suspension of cells with an anti-CD28 antibody agonist. Alternatively, in certain embodiments, the second culture conditions does not include contacting the suspension of cells with a CD3 agonist and/or CD28 agonist. The second culture condition can be maintained, e.g., for between 1 and 5 days (e.g., for 1, 2, 3, 4, or 5 days).

### VII. Methods of Isolation of Expanded T Cells

ICOS^{high} CD4+ T cells can be isolated from populations of cells in which they were generated and/or expanded using standard methods. In some embodiments, an anti-ICOS antibody (see, e.g., below) is used to isolate the cells. In one example, such an antibody is used to label the ICOS^{high} CD4+ T cells, and flow cytometry is used to isolate the cells labeled with the antibody. In another example, an anti-ICOS antibody (see, e.g., below) is bound to a plate or other solid substrate and a cell preparation including ICOS^{high} CD4+ T cells is contacted with the plate or solid substrate. The ICOS^{high} CD4+ T cells will be bound to the plate or solid substrate and can be eluted therefrom after washing.

### VIII. Storage

Any method of storage known in the art suitable for maintaining viability of CD4+ T cells may be used to store ICOS^{high} CD4+ T cells generated according to the methods of the invention. In some embodiments, the stored CD4+ T cells are stored in a cell culture medium. In some embodiments, the stored CD4+ T cells are stored at a concentration of greater than 100,000 cells/mL. In some embodiments, the stored CD4+ T cells are stored at a concentration between 100,000 cells/mL and 100 million cells/mL. In some embodiments, the invention provides a suspension of stored CD4+ T cells obtained, accordingly. Storage methods that can be used in the invention are described, e.g., by Abazari et al., Cell & Gene Therapy Insights 3(10):853-871, 2017. Acceptable methods include, for example, cryopreservation, hypothermic preservation, refrigerated storage at 4° C., lyophilization, alginate encapsulation, and vitrification.

Cryopreservation is a common method for long term storage of cell therapy products. Approaches for cryopreservation are described, e.g., in Coopman et al., StemBook, Harvard Stem Cell Institute, 2014. In various examples of cryopreservation methods, specific cooling regimens are used down to temperatures at or below −80° C. (e.g., at or below −120° C. or at or below −135° C.). These approaches can utilize liquid nitrogen storage or an electric freezer, which allows for specific temperature control. Cryopreserved materials can optionally be shipped on dry ice, in order to ensure a “cold chain” from storage of the materials to shipment to a site for thaw and use.

Hypothermic preservation is a short term preservation method in which cells are held above 0° C., but are effectively “paused” and their metabolism is greatly slowed to the point where they are suspended (for, e.g., a week or so). See, e.g., the following references in regard to hypothermic preservation: Robinson et al., Biotechnology. Lett. 36(2):201-209, 2014; and Correia et al., Stem Cells Transl. Med. 5(5):658-669, 2016.

Refrigeration at 4 C can be used for very short term storage (e.g., same day to about 96 hours). Thus, refrigeration at 4 C is used for essentially fresh product. Storage time can be increased by the use of, e.g., preservative additives, changing cell density, media feeding, and media refreshing, as can be determined by those of skill in the art.

### IX. Pre-Treatment of Patients (Prior to Isolating Immune Cells)

In some embodiments, the methods of the invention include pre-treating patients prior to obtaining their immune cells for use in the generation of ICOS^{high} CD4+ T cells. Such pre-treatment can include, for example, treatment with one or more of the following: chemotherapies; anti-CTLA4 antibodies; agonists of anti-TLR9 (e.g., CpG oligodeoxynucleotides), TLR2/4, TLR7/8, and/or TLR7; radiation; ICOS agonists; tyrosine kinase inhibitors; epigenetic modifiers (e.g., HDAC inhibitors and demethylating agents); IMIDs (immuno-modulatory drugs, e.g., lenalidomide); cytokines (e.g., interferon gamma); oncolytic viruses; vaccines; and CD40 agonists. Specific examples of some of these types of treatments are described below. The patient may receive one or more (e.g., 1, 2, 3, 4, 5, or more) pre-treatment doses or cycles.

### X. Methods of Treatment Using Expanded Cells

ICOS^{high} CD4+ T cells such as those described herein can be used in methods for preventing, improving, and treating diseases including, for example, cancer and infectious diseases (e.g., HIV, tuberculosis, Epstein Barr virus, herpes simplex virus, malaria, or human papilloma virus).

Patients that can be treated as described herein thus include patients having a cancer. The type of cancer can be any type of cancer listed herein or otherwise known in the art. Exemplary types of cancer include, but are not limited to, gastric cancer, breast cancer (e.g., triple negative breast cancer (TNBC)), lung cancer (e.g., non-small cell lung cancer (NSCLC)), melanoma, renal cell carcinoma (RCC), bladder cancer, endometrial cancer, diffuse large B-cell lymphoma (DLBCL), Hodgkin's lymphoma, ovarian cancer, and head and neck squamous cell cancer (HNSCC). Also see the definition of cancer, above, for additional cancer types that can be treated according to the methods of the invention.

Patients that can be treated as described herein include patients who have not previously received a different anti-cancer therapy and patients who have received previous (e.g., 1, 2, 3, 4, 5, or more) doses or cycles of one or more (e.g., 1, 2, 3, 4, 5, or more) of different anti-cancer therapies including, e.g., the pre-treatment described above.

In addition to treatment with ICOS^{high} CD4+ T cells, any of the anti-cancer therapies listed herein (see below) and others known in the art can be used in connection with the methods of the invention. In some embodiments, the one or more additional anti-cancer therapies is two or more anti-cancer therapies. In some embodiments, the one or more additional anti-cancer therapies is three or more anti-cancer therapies. Specific, non-limiting examples of additional anti-cancer therapies that can be used in the invention including, e.g., immunotherapies, chemotherapies, and cancer vaccines, among others, are provided below. In some embodiments, the one or more additional anti-cancer therapies is administered prior to the ICOS^{high} CD4+ T cell therapy. In some embodiments, the one or more additional anti-cancer therapies is administered at the same time as the ICOS^{high} CD4+ T cell therapy. In some embodiments, the one or more additional anti-cancer therapies is administered after the ICOS^{high} CD4+ T cell therapy.

In some embodiments, the ICOS^{high} CD4+ T cell therapy (and/or the one or more additional anti-cancer therapies) is administered to the patient multiple times at regular intervals. These multiple administrations can also be referred to as administration cycles or therapy cycles. In some embodiments, the ICOS^{high} CD4+ T cell therapy (and/or the one or more anti-cancer therapies) is administered to the patient for more than two cycles, more than three cycles, more than four cycles, more than five cycles, more than ten cycles, more than fifteen cycles, or more than twenty cycles.

In some embodiments, the regular interval is a dosage every week, a dosage every two weeks, a dosage every three weeks, a dosage every four weeks, a dosage every five weeks, a dosage every six weeks, a dosage every seven weeks, a dosage every eight weeks, a dosage every nine weeks, a dosage every ten weeks, a dosage every eleven weeks, or a dosage every twelve weeks.

### XI. Exemplary Anti-Cancer Therapies for Use in the Methods of the Invention

As examples, any anti-cancer therapy listed herein or otherwise known in the art, can be used in combination with ICOS^{high} CD4+ T cell therapy as described herein or as a pre-treatment, as described above. Exemplary anti-cancer therapies are described below.

a. Immunotherapies

In some embodiments, the one or more anti-cancer therapies is an immunotherapy. The interaction between cancer and the immune system is complex and multifaceted. See de Visser et al., Nat. Rev. Cancer 624-37, 2006. While many cancer patients appear to develop an anti-tumor immune response, cancers also develop strategies to evade immune detection and destruction. Recently, immunotherapy has been developed for the treatment and prevention of cancer and other disorders. Immunotherapy provides the advantage of cell specificity that other treatment modalities lack. As such, methods for enhancing the efficacy of immune based therapies can be clinically beneficial.

i. Therapeutic Anti-ICOS Antibodies

Therapeutic anti-ICOS antibodies that can be used in the invention include, but are not limited to, humanized antibodies, chimeric antibodies, human antibodies, and antibodies comprising any of the heavy chain and/or light chain CDRs discussed herein. In some embodiments, the antibody is an isolated antibody. In some embodiments, the antibody is a monoclonal antibody. In some embodiments, the anti-ICOS antibody is an anti-ICOS agonist antibody. See WO 2016/154177 and WO 2017/070423, which are each specifically incorporated herein by reference.

In some embodiments, the therapeutic anti-ICOS agonist antibody includes at least one, two, there, four, five, or all six CDRs selected from (a) HCDR1 comprising the amino acid sequence of SEQ ID NO: 5; (b) HCDR2 comprising the amino acid sequence of SEQ ID NO: 6; (c) HCDR3 comprising the amino acid sequence of SEQ ID NO: 7; (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10. In various embodiments, one or more of the CDRs includes a substitution or deletion that does not destroy specific binding to ICOS. In some embodiments, one or more of the CDRs includes 1, 2, 3, or more substitutions, which may optionally comprise substitutions with conservative amino acids. In some embodiments, one or more of the CDRs includes 1, 2, 3, or more deletions.

In some embodiments, the therapeutic anti-ICOS antibody comprises six CDRs including (a) HCDR1 comprising the amino acid sequence of SEQ ID NO: 5; (b) HCDR2 comprising the amino acid sequence of SEQ ID NO: 6; (c) HCDR3 comprising the amino acid sequence of SEQ ID NO: 7; (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10.

In some embodiments, a therapeutic anti-ICOS antibody comprises a heavy chain variable region and a light chain variable region. In some embodiments, a therapeutic anti-ICOS antibody comprises at least one heavy chain comprising a heavy chain variable region and at least a portion of a heavy chain constant region, and at least one light chain comprising a light chain variable region and at least a portion of a light chain constant region. In some embodiments, a therapeutic anti-ICOS antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some embodiments, the heavy chain is the region of the anti-ICOS antibody that comprises the three heavy chain CDRs. In some embodiments, the light chain is the region of the therapeutic anti-ICOS antibody that comprises the three light chain CDRs.

In some embodiments, the therapeutic anti-ICOS antibody comprises at least one, at least two, or all three VH CDR sequences selected from (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7.

In some embodiments, the therapeutic antibody comprises at least one, at least two, or all three VL CDR sequences selected from (a) LCDR1 comprising the amino acid sequence of SEQ ID NO: 8; (b) LCDR2 comprising the amino acid sequence of SEQ ID NO: 9; and (c) LCDR3 comprising the amino acid sequence of SEQ ID NO: 10.

In some embodiments, the therapeutic anti-ICOS antibody comprises (1) a VH domain comprising at least one, at least two, or all three VH CDR sequences selected from (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7; and (II) a VL domain comprising at least one, at least two, or all three VL CDR sequences selected from (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10.

In some embodiments, a therapeutic anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 3. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted, and/or deleted in SEQ ID NO: 3. In some embodiments, substitutions, insertions, or deletions occur in regions outside the CDRs (that is, in the FRs). Optionally, the therapeutic anti-ICOS antibody comprises the VH sequence in SEQ ID NO: 3, including post-translational modifications of that sequence.

In some embodiments, the VH comprises: (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7.

In some embodiments, a therapeutic anti-ICOS antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 4. In some embodiments, a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted, and/or deleted in SEQ ID NO: 4. In some embodiments, the substitutions, insertions, or deletions occur in regions outside the CDRs (that is, in the FRs). Optionally, the therapeutic anti-ICOS antibody comprises the VL sequence in SEQ ID NO: 4, including post-translational modifications of that sequence.

In some embodiments, the VL comprises: (a) LCDR1 comprising the amino acid sequence of SEQ ID NO: 8; (b) LCDR2 comprising the amino acid sequence of SEQ ID NO: 9; and (c) LCDR3 comprising the amino acid sequence of SEQ ID NO: 10.

In some embodiments, a therapeutic anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 3 and a light chain variable domain (VL) having at least 90%, 91%, 92%, 93%, 94%, 95%, 96%, 97%, 98%, 99%, or 100% sequence identity to the amino acid sequence of SEQ ID NO: 4. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted, and/or deleted in SEQ ID NO: 3. In some embodiments, a total of 1 to 10 amino acids have been substituted, inserted, and/or deleted in SEQ ID NO: 4. In some embodiments, substitutions, insertions, or deletions occur in regions outside the CDRs (that is, in the FRs). Optionally, the therapeutic anti-ICOS antibody comprises the VH sequence in SEQ ID NO: 3 and the VL sequence of SEQ ID NO: 4, including post-translational modifications of one or both sequence.

In some embodiments, the therapeutic anti-ICOS antibody comprises (1) a VH domain comprising: (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7; and (11) a VL domain comprising: (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10.

In some embodiments, a therapeutic anti-ICOS antibody comprises a VH as in any of the embodiments provided herein, and a VL as in any of the embodiments provided herein. In some embodiments, the antibody comprises the VH and VL sequences in SEQ ID NO: 3 and SEQ ID NO: 4, respectively, including post-translational modifications of those sequences.

In some embodiments, a therapeutic anti-ICOS antibody comprises a heavy chain comprising the amino acid sequence of SEQ ID NO: 1, or a variant thereof.

In some embodiments, a therapeutic anti-ICOS antibody comprises a light chain comprising the amino acid sequence of SEQ ID NO: 2, or a variant thereof.

In some embodiments, a therapeutic anti-ICOS antibody comprises a heavy chain comprising the amino acid sequence of SEQ ID NO: 1 and a light chain comprising the amino acid sequence of SEQ ID NO: 2, or variants thereof.

In some embodiments, the therapeutic anti-ICOS antibody comprises the six CDRs as described above and binds to ICOS. In some embodiments, the therapeutic anti-ICOS antibody comprises the six CDRs as described above, binds to ICOS and increases the number of Teff cells and/or activates Teff cells and/or decreases the number of Treg cells and/or increases the ratio of Teff cells to Treg cells in a mammal, such as a human. In some embodiments, the Treg cells are CD4+ FoxP3+ T cells. In some embodiments, the Teff cells are CD8+ T cells. In some embodiments, the Teff cells are CD4+ FoxP3− T cells and/or CD8+ T cells.

Exemplary therapeutic anti-ICOS antibodies include, but are not limited to, JTX-2011 (Jounce Therapeutics; US 2016/0304610; WO 2016/154177; WO 2017/070423) and BMS-986226 (Bristol-Myers Squibb).

In general, therapeutic anti-ICOS antibodies can be administered in an amount in the range of about 10 μg/kg body weight to about 100 mg/kg body weight per dose. In some embodiments, therapeutic anti-ICOS antibodies may be administered in an amount in the range of about 50 μg/kg body weight to about 5 mg/kg body weight per dose. In some embodiments, therapeutic anti-ICOS antibodies may be administered in an amount in the range of about 100 μg/kg body weight to about 10 mg/kg body weight per dose. In some embodiments, therapeutic anti-ICOS antibodies may be administered in an amount in the range of about 100 μg/kg body weight to about 20 mg/kg body weight per dose. In some embodiments, therapeutic anti-ICOS antibodies may be administered in an amount in the range of about 0.5 mg/kg body weight to about 20 mg/kg body weight per dose. In some embodiments, anti-ICOS antibodies may be administered in an amount in the range of about 0.05 mg/kg body weight to about 10 mg/kg body weight per dose. In some embodiments, anti-ICOS antibodies may be administered in an amount in the range of about 5 mg/kg body weight or lower, for example less than 4, less than 3, less than 2, or less than 1 mg/kg of the antibody. In specific examples, therapeutic anti-ICOS antibodies are administered at 0.1 mg/kg, 0.3 mg/kg, or 1.0 mg/kg, once every 3, 6, 9, or 12 weeks.

ii. Anti-CTLA-4 Antagonist Antibodies

In some embodiments, the one or more anti-cancer therapies is an anti-CTLA-4 antagonist antibody. An anti-CTLA-4 antagonist antibody refers to an agent capable of inhibiting the activity of cytotoxic T-lymphocyte-associated protein 4 (CTLA-4), thereby activating the immune system. The CTLA-4 antagonist may bind to CTLA-4 and reverse CTLA-4-mediated immunosuppression. A non-limiting exemplary anti-CTLA-4 antibody is ipilimumab (YERVOY®, BMS), which may be administered according to methods known in the art, e.g., as approved by the US FDA. For example, ipilimumab may be administered in the amount of 3 mg/kg intravenously over 90 minutes every three weeks for a total of 4 doses (unresectable or metastatic melanoma); or at 10 mg/kg intravenously over 90 minutes every three weeks for a total of 4 doses, followed by 10 mg/kg every 12 weeks for up to 3 years or until documented recurrence or unacceptable toxicity (adjuvant melanoma).

iii. OX40 Agonist Antibodies

In some embodiments, the one or more anti-cancer therapies is an agonist anti-OX40 antibody. An OX40 agonist antibody refers to an agent that induces the activity of OX40, thereby activating the immune system and enhancing anti-tumor activity. Non-limiting, exemplary agonist anti-OX40 antibodies are Medi6469, Medlmmune, and MOXR0916/RG7888, Roche. These antibodies may be administered according to methods and in regimens determined to be appropriate by those of skill in the art.

iv. PD-1 Therapies

In some embodiments, the one or more anti-cancer therapies is a PD-1 therapy. A PD-1 therapy encompasses any therapy that modulates PD-1 binding to PD-L1 and/or PD-L2. PD-1 therapies may, for example, directly interact with PD-1 and/or PD-L1. In some embodiments, a PD-1 therapy includes a molecule that directly binds to and/or influences the activity of PD-1. In some embodiments, a PD-1 therapy includes a molecule that directly binds to and/or influences the activity of PD-L1. Thus, an antibody that binds to PD-1 or PD-L1 and blocks the interaction of PD-1 to PD-L1 is a PD-1 therapeutic. When a desired subtype of PD-1 therapy is intended, it will be designated by the phrase “PD-1 specific” for a therapy involving a molecule that interacts directly with PD-1, or “PD-L1 specific” for a molecule that interacts directly with PD-L1, as appropriate. Unless designated otherwise, all disclosure contained herein regarding PD-1 therapy applies to PD-1 therapy generally, as well as PD-1 specific and/or PD-L1 specific therapies.

Non-limiting, exemplary PD-1 therapies include nivolumab (OPDIVO®, BMS-936558, MDX-1106, ONO-4538); pidilizumab, lambrolizumab/pembrolizumab (KEYTRUDA, MK-3475); BGB-A317, tislelizumab (BeiGene/Celgene); durvalumab (anti-PD-L1 antibody, MEDI-4736; AstraZeneca/Medlmmune); RG-7446; avelumab (anti-PD-L1 antibody; MSB-0010718C; Pfizer); AMP-224; BMS-936559 (anti-PD-L1 antibody); AMP-514; MDX-1105; A B-011; anti-LAG-3/PD-1; spartalizumab (CoStim/Novartis); anti-PD-1 antibody (Kadmon Pharm.); anti-PD-1 antibody (Immunovo); anti-TEVI-3/PD-1 antibody (AnaptysBio); anti-PD-L1 antibody (CoStim/Novartis); RG7446/MPDL3280A (anti-PD-L1 antibody, Genentech/Roche); KD-033 (Kadmon Pharm.); AGEN-2034 (Agenus); STI-A1010; STI-A1110; TSR-042; atezolizumab (TECENTRIQ™); and other antibodies that are directed against programmed death-1 (PD-1) or programmed death ligand 1 (PD-L1).

PD-1 therapies are administered according to regimens that are known in the art, e.g., US FDA-approved regimens. In one example, nivolumab is administered as an intravenous infusion over 60 minutes in the amount of 240 mg every two weeks (unresectable or metastatic melanoma, adjuvant treatment for melanoma, non-small cell lung cancer (NSCLC), advanced renal cell carcinoma, locally advanced renal cell carcinoma, MSI-H or dMMR metastatic colorectal cancer, and hepatocellular carcinoma) or in the amount of 3 mg/kg every three weeks (classical Hodgkin lymphoma; recurrent or metastatic squamous cell carcinoma of the head and neck). In another example, pembrolizumab is administered by intravenous infusion over 30 minutes in the amount of 200 mg, once every three weeks. In another example, atezolizumab is administered by intravenous infusion over 60 minutes in the amount of 1200 mg every three weeks. In another example, avelumab is administered by intravenous infusion over 60 minutes in the amount of 10 mg/kg every two weeks. In another example, durvalumab is administered by intravenous infusion over 60 minutes in the amount of 10 mg/kg every two weeks.

v. TIGIT Antagonists

In some embodiments, the one or more anti-cancer therapies is TIGIT antagonist. A TIGIT antagonist refers to an agent capable of antagonizing or inhibiting the activity of T-cell immunoreceptor with Ig and ITIM domains (TIGIT), thereby reversing TIGIT-mediated immunosuppression. A non-limiting exemplary TIGIT antagonist is BMS-986207 (Bristol-Myers Squibb/Ono Pharmaceuticals). These agents may be administered according to methods and in regimens determined to be appropriate by those of skill in the art.

vi. IDO Inhibitors

In some embodiments, the one or more anti-cancer therapies is an IDO inhibitor. An IDO inhibitor refers to an agent capable of inhibiting the activity of indoleamine 2,3-dioxygenase (IDO) and thereby reversing IDO-mediated immunosuppression. The IDO inhibitor may inhibit IDO1 and/or ID02 (INDOL1). An IDO inhibitor may be a reversible or irreversible IDO inhibitor. A reversible IDO inhibitor is a compound that reversibly inhibits IDO enzyme activity either at the catalytic site or at a non-catalytic site while an irreversible IDO inhibitor is a compound that irreversibly inhibits IDO enzyme activity by forming a covalent bond with the enzyme. Non-limiting exemplary IDO inhibitors are described, e.g., in US 2016/0060237; and US 2015/0352206. Non-limiting exemplary IDO inhibitors include Indoximod (New Link Genetics), INCB024360 (Incyte Corp), 1-methyl-D-tryptophan (New Link Genetics), and GDC-0919/navoximod (Genentech/New Link Genetics). These agents may be administered according to methods and in regimens determined to be appropriate by those of skill in the art.

vii. RORγ Agonists

In some embodiments, the one or more anti-cancer therapies is a RORγ agonist. RORγ agonists refer to an agent capable of inducing the activity of retinoic acid-related orphan receptor gamma (RORγ), thereby decreasing immunosuppressive mechanisms. Non-limiting exemplary RORγ agonists include, but are not limited to, LYC-55716 (Lycera/Celgene) and INV-71 (Innovimmune). These agents may be administered according to methods and in regimens determined to be appropriate by those of skill in the art.

b. Chemotherapies

In some embodiments, the one or more anti-cancer therapies is a chemotherapeutic agent. Exemplary chemotherapeutic agents that can be used include, but are not limited to, capecitabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, ABRAXA E® (protein-bound paclitaxel), pemetrexed, vinorelbine, vincristine, erlotinib, afatinib, gefitinib, crizotinib, dabrafenib, trametinib, vemurafenib, and cobimetanib. These agents may be administered according to methods and in regimens determined to be appropriate by those of skill in the art.

c. Cancer Vaccines

In some embodiments, the one or more anti-cancer therapies is a cancer vaccine. Cancer vaccines have been investigated as a potential approach for antigen transfer and activation of dendritic cells. In particular, vaccination in combination with immunologic checkpoints or agonists for co-stimulatory pathways have shown evidence of overcoming tolerance and generating increased anti-tumor response. A range of cancer vaccines have been tested that employ different approaches to promoting an immune response against the tumor (see, e.g., Emens L A, Expert Opin Emerg Drugs 13(2): 295-308 (2008)). Approaches have been designed to enhance the response of B cells, T cells, or professional antigen-presenting cells against tumors. Exemplary types of cancer vaccines include, but are not limited to, peptide-based vaccines that employ targeting distinct tumor antigens, which may be delivered as peptides/proteins or as genetically-engineered DNA vectors, viruses, bacteria, or the like; and cell biology approaches, for example, for cancer vaccine development against less well-defined targets, including, but not limited to, vaccines developed from patient-derived dendritic cells, autologous tumor cells or tumor cell lysates, allogeneic tumor cells, and the like.

Exemplary cancer vaccines include, but are not limited to, dendritic cell vaccines, oncolytic viruses, tumor cell vaccines, etc. In some embodiments, such vaccines augment the anti-tumor response. Examples of cancer vaccines also include, but are not limited to, MAGE3 vaccine (e.g., for melanoma and bladder cancer), MUC1 vaccine (e.g., for breast cancer), EGFRv3 (such as Rindopepimut, e.g., for brain cancer, including glioblastoma multiforme), or ALVAC-CEA (e.g., for CEA+ cancers).

Non-limiting exemplary cancer vaccines also include Sipuleucel-T, which is derived from autologous peripheral-blood mononuclear cells (PBMCs) that include antigen-presenting cells (see, e.g., Kantoff P W et al., N Engl J Med. 363:411-22 (2010)). In Sipuleucel-T generation, the patient's PBMCs are activated ex vivo with PA2024, a recombinant fusion protein of prostatic acid phosphatase (a prostate antigen) and granulocyte-macrophage colony-stimulating factor (an immune-cell activator). Another approach to a candidate cancer vaccine is to generate an immune response against specific peptides mutated in tumor tissue, such as melanoma (see, e.g., Carreno et al., Science 348:6236, 2015). Such mutated peptides may, in some embodiments, be referred to as neoantigens. As a non-limiting example of the use of neoantigens in tumor vaccines, neoantigens in the tumor predicted to bind the major histocompatibility complex protein HLA-A*02:01 are identified for individual patients with a cancer, such as melanoma. Dendritic cells from the patient are matured ex vivo, then incubated with neoantigens. The activated dendritic cells are then administered to the patient. In some embodiments, following administration of the cancer vaccine, robust T-cell immunity against the neoantigen is detectable.

In some such embodiments, the cancer vaccine is developed using a neoantigen. In some embodiments, the cancer vaccine is a DNA vaccine. In some embodiments, the cancer vaccine is an engineered virus comprising a cancer antigen, such as PROSTVAC (rilimogene galvacirepvec/rilimogene glafolivec). In some embodiments, the cancer vaccine comprises engineered tumor cells, such as GVAX, which is a granulocyte-macrophage colony-stimulating factor (GM-CSF) gene-transfected tumor cell vaccine (see, e.g., Nemunaitis, Expert Rev. Vaccines 4:259-274, 2005).

The vaccines may be administered according to methods and in regimens determined to be appropriate by those of skill in the art.

d. Additional Exemplary Anti-Cancer Therapies

Further non-limiting, exemplary anti-cancer therapies include Luspatercept (Acceleron Pharma/Celgene); Motolimod (Array BioPharma/Celgene/VentiRx Pharmaceuticals/Ligand); GI-6301 (Globelmmune/Celgene/NantWorks); GI-6200 (Globelmmune/Celgene/NantWorks); BLZ-945 (Celgene/Novartis); ARRY-382 (Array BioPharma/Celgene), or any of the anti-cancer therapies provided in Table 2. These agents may be administered according to methods and in regimens determined to be appropriate by those of skill in the art. In some embodiments, the one or more anti-cancer therapies includes surgery and/or radiation therapy. Accordingly, the anti-cancer therapies can optionally be utilized in the adjuvant or neoadjuvant setting.

e. Combinations

In various embodiments, the anti-cancer treatment (in addition to ICOS^{high} CD4+ T cell therapy) administered to a patient is a combination of one or more (e.g., two, three, or more) additional anti-cancer treatments including, e.g., one or more of the additional anti-cancer treatments listed above or elsewhere herein.

In various examples, in addition to the ICOS^{high} CD4+ T cell therapy, an anti-ICOS agonist antibody (e.g., an antibody described above, such as JTX-2011) is administered in combination with another immunotherapy (see, e.g., above). In one example, an anti-ICOS agonist antibody (e.g., an antibody described above, such as JTX-2011) is administered in combination with a PD-1 therapy (e.g., a PD-1 therapy listed above). Thus, the invention includes, in various examples, administration of an anti-ICOS agonist antibody (e.g., JTX-2011) in combination with one or more of nivolumab, pidilizumab, lambrolizumab/pembrolizumab, BGB-A317, tislelizumab, durvalumab, RG-7446, avelumab, AMP-224, BMS-936559, AMP-514, MDX-1105, A B-011, anti-LAG-3/PD-1, spartalizumab (CoStim/Novartis); anti-PD-1 antibody (Kadmon Pharm.); anti-PD-1 antibody (Immunovo); anti-TEVI-3/PD-I antibody (AnaptysBio); anti-PD-L1 antibody (CoStim/Novartis); RG7446/MPDL3280A, KD-033 (Kadmon Pharm.); AGEN-2034 (Agenus), STI-A1010, STI-A1110, TSR-042, atezolizumab, and other antibodies that are directed against programmed death-1 (PD-1) or programmed death ligand 1 (PD-L1). In one specific example, JTX-2011 is administered with nivolumab.

Optionally, the combinations noted above further include one or more additional anti-cancer agents (e.g., immunotherapies). Accordingly, the combinations noted above can optionally include one or more of an anti-CTLA-4 antagonist antibody (e.g., ipilimumab), an anti-OX40 antibody (e.g., Medi6469), or MOXR0916/RG7888), a TIGIT antagonist (e.g., BMS-986207), an IDO inhibitor (e.g., indoximod, INCB024360, 1-methyl-D-tryptophan, or GDC-0919/navoximod), an RORγ agonist (e.g., LYC-55716 and INV-71), or a chemotherapeutic agent (see, e.g., above), or a cancer vaccine (see, e.g., above).

In other examples, a combination of the invention includes an anti-ICOS agonist antibody (e.g., an antibody described above, such as JTX-2011) and one or more of an anti-CTLA-4 antagonist antibody (e.g., ipilimumab), an anti-OX40 antibody (e.g., Medi6469), or MOXR0916/RG7888), a TIGIT antagonist (e.g., BMS-986207), an IDO inhibitor (e.g., indoximod, INCB024360, 1-methyl-D-tryptophan, or GDC-0919/navoximod), an RORγ agonist (e.g., LYC-55716 and INV-71), or a chemotherapeutic agent (see, e.g., above), or a cancer vaccine (see, e.g., above).

In various examples, the components of a combination are administered according to dosing regimens described herein (e.g., US FDA-approved dosing regimens; see above), or using other regimens determined to be appropriate by those of skill in the art.

### XII. Pharmaceutical Compositions and Dosing

Compositions including ICOS^{high} CD4+ T cells (or one or more additional anti-cancer therapies as described herein) are provided in formulations with a wide variety of pharmaceutically acceptable carriers, as determined to be appropriate by those of skill in the art (see, for example, Gennaro, Remington: The Science and Practice of Pharmacy with Facts and Comparisons: Drugfacts Plus, 20^{th} ed. (2003); Ansel et al., Pharmaceutical Dosage Forms and Drug Delivery Systems, 7^{th} ed., Lippincott, Williams and Wikins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

Anti-cancer therapies are administered in the practice of the methods of the present invention as is known in the art (e.g., according to FDA-approved regimens) or as indicated elsewhere herein (see, e.g., above). In some embodiments, anti-cancer therapies of the invention are administered in amounts effective for treatment of cancer. The therapeutically effective amount is typically dependent on the weight of the subject being treated, his or her physical or health condition, the extensiveness of the condition to be treated, the age of the subject being treated, pharmaceutical formulation methods, and/or administration methods (e.g., administration time and administration route).

In some embodiments, anti-cancer therapies can be administered in vivo by various routes, including, but not limited to, intravenous, intra-arterial, parenteral, intratumoral, intraperitoneal, or subcutaneous. The appropriate formulation and route of administration can be selected by those of skill in the art according to the intended application.

### XIII. Examples

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Strong Stimulation of TCR in PBMC Samples is Sufficient to Drive Induction of ICOS Hi Cell Phenotype In Vitro

10⁵ healthy donor PBMCs from three donors were distributed to the wells of a 48-well plate and incubated with decreasing concentrations of soluble OKT-3 (anti-CD3) in AIM-V medium (Thermo A3830801) supplemented with 5% human AB serum (Sigma H4522) and 1% antibiotic antimycotic solution (Sigma A5955). Cells were incubated for 72 hours at 37° C., then stained and fixed.

A dose-response relationship emerged between the amount of added OKT3 and the emergence of an ICOS hi, CD4 positive cell population.

### Example 2. Antigen Stimulation Followed by Re-Stimulation with an ICOS Agonist Results in Activation of ICOS Hi, CD4+ T-Cells

Peripheral blood mononuclear cells were acquired from whole blood from three healthy donors. Cells were plated in duplicate in a 48 well plate format and incubated for 72 hours at 37° C. in media containing Th1 skewing cytokines (IL-12, IL-2 and anti-IL-4), tetanus toxoid, and soluble anti-CD28. At the conclusion of the three-day incubation, stimulus was removed and cells were washed. Cells then received soluble JTX-2011 or no additional stimulation. Concurrently, Brefeldin A was applied to all wells for 4 hours and intracellular cytokine levels were assessed using flow cytometry.

In the presence of agonism of the ICOS, significant Th1 cytokine responses were observed. These data suggests that the combination of exposure to a relevant TCR antigen followed by ICOS agonism can be sufficient to activate T-cells.

### Example 3. Expansion of ICOShi CD4+ T Cell Populations

3.1

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by OKT-3+IL-2 to induce ICOS expression among CD4+ T cells. All supplements were delivered in soluble format.

Cells were incubated for 3 days at 37° C., then transferred to new wells containing fresh media and all supplements used for initial stimulation. Media was further supplemented by Nivolumab (anti-PD-1) to maintain ICOS expression and prevent exhaustion among CD4+ T cells. All supplements were delivered in soluble format.

Cells were incubated for another 4 days at 37° C., then transferred to new wells containing fresh media and all supplements used for initial stimulation and ICOS maintenance. Cells were then incubated for another 3 days at 37° C., then stained and fixed. At the end of 10 total days of incubation, cells had expanded by a minimum of 24-fold.

3.2

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by OKT-3+IL-2+ICOS-L to induce ICOS expression among CD4+ T cells. All supplements were delivered in soluble format, with the exception of ICOS-L which was previously coated onto the culture plate (plate-bound format).

Cells were incubated for 3 days at 37° C., then transferred to new wells containing fresh media and all supplements used for initial stimulation. Media was further supplemented by Nivolumab (anti-PD-1) to maintain ICOS expression and prevent exhaustion among CD4+ T cells. All supplements were delivered in soluble format, with the exception of ICOS-L which was previously coated onto the culture plate (plate-bound format).

Cells were incubated for another 4 days at 37° C., then transferred to new wells containing fresh media and all supplements used for initial stimulation and ICOS maintenance. Cells were then incubated for another 3 days at 37° C., then stained and fixed. At the end of 10 total days of incubation, cells had expanded by a minimum of 24-fold.

3.3

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by IL-2+IL-12+anti-IL-4+Stemcell ImmunoCult human CD3/CD28 T cell activator (Cat #10971) to induce ICOS expression among CD4+ T cells. All supplements were delivered in soluble format.

Cells were incubated for 3 days at 37° C., then transferred to new wells containing fresh media and all supplements used for initial stimulation. Media was further supplemented by Nivolumab (anti-PD-1) to maintain ICOS expression and prevent exhaustion among CD4+ T cells. All supplements were delivered in soluble format.

Cells were incubated for another 4 days at 37° C., then transferred to new wells containing fresh media and all supplements used for initial stimulation and ICOS maintenance. Cells were then incubated for another 3 days at 37° C., then stained and fixed. At the end of 10 total days of incubation, cells had expanded by a minimum of 24-fold.

3.4

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by IL-2+IL-12+anti-IL-4+Stemcell ImmunoCult human CD3/CD28 T cell activator (Cat #10971)+ICOS-L to induce ICOS expression among CD4+ T cells. All supplements were delivered in soluble format, with the exception of ICOS-L which was previously coated onto the culture plate (plate-bound format).

Cells were incubated for 3 days at 37° C., then transferred to new wells containing fresh media and all supplements used for initial stimulation. Media was further supplemented by Nivolumab (anti-PD-1) to maintain ICOS expression and prevent exhaustion among CD4+ T cells. All supplements were delivered in soluble format, with the exception of ICOS-L which was previously coated onto the culture plate (plate-bound format).

Cells were incubated for another 4 days at 37° C., then transferred to new wells containing fresh media and all supplements used for initial stimulation and ICOS maintenance. Cells were then incubated for another 3 days at 37° C., then stained and fixed. At the end of 10 total days of incubation, cells had expanded by a minimum of 24-fold.

3.5

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by OKT-3+IL-2 to induce ICOS expression among CD4+ T cells. All supplements were delivered in soluble format.

Cells were incubated for 3 days at 37° C., then transferred to new wells containing fresh media and all supplements used for initial stimulation. Media was further supplemented by Ipilimumab (anti-CTLA-4) to maintain ICOS expression and prevent exhaustion among CD4+ T cells. All supplements were delivered in soluble format.

Cells were incubated for another 4 days at 37° C., then transferred to new wells containing fresh media and all supplements used for initial stimulation and ICOS maintenance. Cells were then incubated for another 3 days at 37° C., then stained and fixed. At the end of 10 total days of incubation, cells had expanded by a minimum of 24-fold.

3.6

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by OKT-3+IL-2+ICOS-L to induce ICOS expression among CD4+ T cells. All supplements were delivered in soluble format, with the exception of ICOS-L which was previously coated onto the culture plate (plate-bound format).

Cells were incubated for 3 days at 37° C., then transferred to new wells containing fresh media and all supplements used for initial stimulation. Media was further supplemented by Ipilimumab (anti-CTLA-4) to maintain ICOS expression and prevent exhaustion among CD4+ T cells. All supplements were delivered in soluble format, with the exception of ICOS-L which was previously coated onto the culture plate (plate-bound format).

Cells were incubated for another 4 days at 37° C., then transferred to new wells containing fresh media and all supplements used for initial stimulation and ICOS maintenance. Cells were then incubated for another 3 days at 37° C., then stained and fixed. At the end of 10 total days of incubation, cells had expanded by a minimum of 24-fold.

3.7

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by IL-2+IL-12+anti-IL-4+Stemcell ImmunoCult human CD3/CD28 T cell activator (Cat #10971) to induce ICOS expression among CD4+ T cells. All supplements were delivered in soluble format.

Cells were incubated for 3 days at 37° C., then transferred to new wells containing fresh media and all supplements used for initial stimulation. Media was further supplemented by Ipilimumab (anti-CTLA-4) to maintain ICOS expression and prevent exhaustion among CD4+ T cells. All supplements were delivered in soluble format.

Cells were incubated for another 4 days at 37° C., then transferred to new wells containing fresh media and all supplements used for initial stimulation and ICOS maintenance. Cells were then incubated for another 3 days at 37° C., then stained and fixed. At the end of 10 total days of incubation, cells had expanded by a minimum of 24-fold.

3.8

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by IL-2+IL-12+anti-IL-4+Stemcell ImmunoCult human CD3/CD28 T cell activator (Cat #10971)+ICOS-L to induce ICOS expression among CD4+ T cells. All supplements were delivered in soluble format, with the exception of ICOS-L which was previously coated onto the culture plate (plate-bound format).

Cells were incubated for 3 days at 37° C., then transferred to new wells containing fresh media and all supplements used for initial stimulation. Media was further supplemented by Ipilimumab (anti-CTLA-4) to maintain ICOS expression and prevent exhaustion among CD4+ T cells. All supplements were delivered in soluble format, with the exception of ICOS-L which was previously coated onto the culture plate (plate-bound format).

Cells were incubated for another 4 days at 37° C., then transferred to new wells containing fresh media and all supplements used for initial stimulation and ICOS maintenance. Cells were then incubated for another 3 days at 37° C., then stained and fixed. At the end of 10 total days of incubation, cells had expanded by a minimum of 24-fold.

3.9

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by OKT-3+IL-2 to induce ICOS expression among CD4+ T cells. All supplements were delivered in soluble format.

Cells were incubated for 3 days at 37° C., then transferred to new wells containing fresh media and all supplements used for initial stimulation, except OKT-3, to maintain ICOS expression and prevent exhaustion among CD4+ T cells. All supplements were delivered in soluble format.

Cells were incubated for another 4 days at 37° C., then transferred to new wells containing fresh media and all supplements used for initial stimulation and ICOS maintenance, including OKT-3. Cells were then incubated for another 3 days at 37° C., then stained and fixed. At the end of 10 total days of incubation, cells had expanded by a minimum of 24-fold.

3.10

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by OKT-3+IL-2+ICOS-L to induce ICOS expression among CD4+ T cells. All supplements were delivered in soluble format, with the exception of ICOS-L which was previously coated onto the culture plate (plate-bound format).

Cells were incubated for 3 days at 37° C., then transferred to new wells containing fresh media and all supplements used for initial stimulation, except OKT-3, to maintain ICOS expression and prevent exhaustion among CD4+ T cells. All supplements were delivered in soluble format, with the exception of ICOS-L which was previously coated onto the culture plate (plate-bound format).

Cells were incubated for another 4 days at 37° C., then transferred to new wells containing fresh media and all supplements used for initial stimulation and ICOS maintenance, including OKT-3. Cells were then incubated for another 3 days at 37° C., then stained and fixed. At the end of 10 total days of incubation, cells had expanded by a minimum of 24-fold.

3.11

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by IL-2+IL-12+anti-IL-4+Stemcell ImmunoCult human CD3/CD28 T cell activator (Cat #10971) to induce ICOS expression among CD4+ T cells. All supplements were delivered in soluble format.

Cells were incubated for 3 days at 37° C., then transferred to new wells containing fresh media and all supplements used for initial stimulation, except Stemcell ImmunoCult human CD3/CD28 T cell activator, to maintain ICOS expression and prevent exhaustion among CD4+ T cells. All supplements were delivered in soluble format.

Cells were incubated for another 4 days at 37° C., then transferred to new wells containing fresh media and all supplements used for initial stimulation and ICOS maintenance, including Stemcell ImmunoCult human CD3/CD28 T cell activator. Cells were then incubated for another 3 days at 37° C., then stained and fixed. At the end of 10 total days of incubation, cells had expanded by a minimum of 24-fold.

3.12

Healthy donor PBMCs were distributed to the wells of a 48-well plate at a density of approximately 3×10⁵ cells per well in 500 uL AIM-V medium (Thermo A3830801) supplemented with 5% human AB serum (Sigma H4522) and 1% antibiotic antimycotic solution (Sigma A5955).

Culture media was further supplemented by IL-2+IL-12+anti-IL-4+Stemcell ImmunoCult human CD3/CD28 T cell activator (Cat #10971)+ICOS-L to induce ICOS expression among CD4+ T cells. All supplements were delivered in soluble format, with the exception of ICOS-L which was previously coated onto the culture plate (plate-bound format).

Cells were incubated for 3 days at 37° C., then transferred to new wells containing fresh media and all supplements used for initial stimulation, except Stemcell ImmunoCult human CD3/CD28 T cell activator, to maintain ICOS expression and prevent exhaustion among CD4+ T cells. All supplements were delivered in soluble format, with the exception of ICOS-L which was previously coated onto the culture plate (plate-bound format).

Cells were incubated for another 4 days at 37° C., then transferred to new wells containing fresh media and all supplements used for initial stimulation and ICOS maintenance, including Stemcell ImmunoCult human CD3/CD28 T cell activator. Cells were then incubated for another 3 days at 37° C., then stained and fixed. At the end of 10 total days of incubation, cells had expanded by a minimum of 24-fold.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

TABLE 2

| Anti-Cancer | | Anti-Cancer | |
|---|---|---|---|
| Therapeutic | Target Name | Therapeutic | Target Name |
| BMS-986179 | 5′-nucleotidase, ecto | imalumab | macrophage migration |
| | (CD73) | | inhibitory factor |
| | | | (glycosylation-inhibiting |
| | | | factor) |
| pTVG-HP | acid phosphatase, prostate | OSE-2101 | major histocompatibility |
| | | | complex, class I, A |
| sipuleucel-T | acid phosphatase, prostate | andecaliximab | matrix metallopeptidase 9 |
| | | | (gelatinase B, 92 kDa |
| | | | gelatinase, 92 kDa type IV |
| | | | collagenase) |
| CX-2009 | activated leukocyte cell | anti-MAGE-A3 | melanoma antigen family A, 3 |
| | adhesion molecule | TCR, Kite Pharma | |
| luspatercept | activin A receptor type II- | KITE-718 | melanoma antigen family A, 3 |
| | like 1 | | |
| CPI-444 | adenosine A2a receptor | biropepimut-S | melanoma antigen family A, 3 |
| NGR-TNF | alanyl (membrane) | rituximab | membrane-spanning 4- |
| | aminopeptidase | biosimilar, Pfizer | domains, subfamily A, |
| | | | member 1 |
| CB-1158 | arginase 1 | rituximab | membrane-spanning 4- |
| | arginase 2 | biosimilar, Dr. | domains, subfamily A, |
| | | Reddy′s | member 1 |
| BA3011 | AXL receptor tyrosine | rituximab | membrane-spanning 4- |
| | kinase | biosimilar, Sandoz | domains, subfamily A, |
| | | | member 1 |
| AXL-107-MMAE | AXL receptor tyrosine | rituximab | membrane-spanning 4- |
| | kinase | biosimilar, Celltrion | domains, subfamily A, |
| | | | member 1 |
| CCT301-38 | AXL receptor tyrosine | rituximab | membrane-spanning 4- |
| | kinase | biosimilar, Archigen | domains, subfamily A, |
| | RAR-related orphan | Biotech | member 1 |
| | receptor A | | |
| SurVaxM | baculoviral IAP repeat | rituximab | membrane-spanning 4- |
| | containing 5 | biosimilar, Innovent | domains, subfamily A, |
| | | Biologies | member 1 |
| NY-ESO-1 TCR, | cancer/testis antigen 1 | MB-106 | membrane-spanning 4- |
| Adaptimmune | | | domains, subfamily A, |
| | | | member 1 |
| CDX-1401 | cancer/testis antigen 1 | ibritumomab | membrane-spanning 4- |
| | lymphocyte antigen 75 | tiuxetan | domains, subfamily A, |
| | | | member 1 |
| ETBX-011 | carcinoembryonic antigen- | rituximab | membrane-spanning 4- |
| | related cell adhesion | | domains, subfamily A, |
| | molecule 5 | | member 1 |
| GI-6207 | carcinoembryonic antigen- | ublituximab | membrane-spanning 4- |
| | related cell adhesion | | domains, subfamily A, |
| | molecule 5 | | member 1 |
| falimarev + | carcinoembryonic antigen- | rituximab | membrane-spanning 4- |
| inalimarev | related cell adhesion | biosimilar, | domains, subfamily A, |
| | molecule 5 | Allergan/Amgen | member 1 |
| | mucin 1, cell surface | | |
| | associated | | |
| labetuzumab | carcinoembryonic antigen- | ofatumumab | membrane-spanning 4- |
| govitecan | related cell adhesion | | domains, subfamily A, |
| | molecule 5 | | member 1 |
| | topoisomerase (DNA) I | | |
| coltuximab | CD19 molecule | ocaratuzumab | membrane-spanning 4- |
| ravtansine | | | domains, subfamily A, |
| | | | member 1 |
| denintuzumab | CD19 molecule | veltuzumab | membrane-spanning 4- |
| mafodotin | | | domains, subfamily A, |
| | | | member 1 |
| axicabtagene | CD19 molecule | obinutuzumab | membrane-spanning 4- |
| ciloleucel | | | domains, subfamily A, |
| | | | member 1 |
| CIK-CAR.CD19 | CD19 molecule | rituximab and | membrane-spanning 4- |
| | | hyaluronidase | domains, subfamily A, |
| | | human | member 1 |
| JCAR014 | CD19 molecule | anetumab | mesothelin |
| | | ravtansine | |
| lisocabtagene | CD19 molecule | amatuximab | mesothelin |
| maraleucel | | | |
| tisagenlecleucel | CD19 molecule | emibetuzumab | met proto-oncogene |
| MOR-208 | CD19 molecule | binimetinib | mitogen-activated protein |
| | | | kinase kinase 1 |
| | | | mitogen-activated protein |
| | | | kinase kinase 2 |
| inebilizumab | CD19 molecule | SAR566658 | mucin 1, cell surface |
| | | | associated |
| AUTO3, Autolus | CD19 molecule | Cvac, Prima | mucin 1, cell surface |
| | CD22 molecule | Biomed | associated |
| DT2219ARL | CD19 molecule | TG4010 | mucin 1, cell surface |
| | CD22 molecule | | associated |
| | | | interleukin 2 receptor, alpha |
| blinatumomab | CD19 molecule | oregovomab | mucin 16, cell surface |
| | CD3e molecule, epsilon | | associated |
| | (CD3-TCR complex) | | |
| samalizumab | CD200 molecule | methionine | opioid growth factor receptor |
| | | enkephalin based | |
| | | immunotherapy | |
| inotuzumab | CD22 molecule | olaratumab | platelet-derived growth factor |
| ozogamicin | | | receptor, alpha polypeptide |
| 90Y- | CD22 molecule | enfortumab vedotin | poliovirus receptor-related 4 |
| epratuzumab | | | |
| tetraxetan | | | |
| epratuzumab | CD22 molecule | ProstAtak, | polymerase (DNA directed), |
| | | Advantagene | alpha 1, catalytic subunit |
| ontuxizumab | CD248 molecule, | PancAtak, | polymerase (DNA directed), |
| | endosialin | Advantagene | alpha 1, catalytic subunit |
| varlilumab | CD27 molecule | aglatimagene | polymerase (DNA directed), |
| | | besadenovec | alpha 1, catalytic subunit |
| durvalumab | CD274 molecule | IMC-gp100 | premelanosome protein |
| avelumab | CD274 molecule | cemiplimab | programmed cell death 1 |
| atezolizumab | CD274 molecule | AGEN2034 | programmed cell death 1 |
| CX-072 | CD274 molecule | nivolumab | programmed cell death 1 |
| enoblituzumab | CD276 molecule | pembrolizumab | programmed cell death 1 |
| omburtamab | CD276 molecule | spartalizumab | programmed cell death 1 |
| AlloStim, | CD28 molecule | BGB-A317 | programmed cell death 1 |
| Immunovative | | | |
| Therapies | | | |
| gemtuzumab | CD33 molecule | genolimzumab | programmed cell death 1 |
| ozogamicin | | | |
| lintuzumab- | CD33 molecule | JNJ-63723283 | programmed cell death 1 |
| Ac225 | | | |
| BI 836858 | CD33 molecule | MEDI0680 | programmed cell death 1 |
| naratuximab | CD37 molecule | thymalfasin | prothymosin, alpha |
| emtansine | | | |
| lutetium (177Lu) | CD37 molecule | LYC-55716 | RAR-related orphan receptor |
| lilotomab | | | C |
| satetraxetan | | | |
| otlertuzumab | CD37 molecule | cirmtuzumab | receptor tyrosine kinase-like |
| | | | orphan receptor 1 |
| daratumumab | CD38 molecule | VX15/2503 | sema domain, |
| | | | immunoglobulin domain (Ig), |
| | | | transmembrane domain (TM) |
| | | | and short cytoplasmic |
| | | | domain, (semaphorin) 4D |
| isatuximab | CD38 molecule | elotuzumab | SLAM family member 7 |
| TAK-573 | CD38 molecule | indatuximab | syndecan 1 |
| | | ravtansine | |
| A-dmDT390- | CD3e molecule, epsilon | BMS-986207 | T-cell immunoreceptor with Ig |
| bisFv (UCHT1) | (CD3-TCR complex) | | and ITIM domains |
| APX005M | CD40 molecule, TNF | tertomotide | telomerase reverse |
| | receptor superfamily | | transcriptase |
| | member 5 | | |
| Hu5F9-G4 | CD47 molecule | Toca 511 + Toca | thymidylate synthetase |
| | | FC | |
| TI-061 | CD47 molecule | APS001F | thymidylate synthetase |
| milatuzumab | CD74 molecule, major | JCARH125 | TNF receptor superfamily |
| | histocompatibility complex, | | member 17 |
| | class II invariant chain | | |
| polatuzumab | CD79b molecule, | bb2121 | TNF receptor superfamily |
| vedotin | immunoglobulin-associated | | member 17 |
| | beta | | |
| mogamulizumab | chemokine (C-C motif) | AUTO2, Autolus | TNF receptor superfamily |
| | receptor 4 | | member 17 |
| | | | TNF receptor superfamily |
| | | | member 13B |
| BL-8040 | chemokine (C-X-C motif) | OPN-305 | toll-like receptor 2 |
| | receptor 4 | | |
| X4P-001 | chemokine (C-X-C motif) | rintatolimod | toll-like receptor 3 |
| | receptor 4 | | |
| ulocuplumab | chemokine (C-X-C motif) | poly-ICLC | toll-like receptor 3 |
| | receptor 4 | | |
| claudiximab | claudin 18 | ID-G100 | toll-like receptor 4 |
| ALT-836 | coagulation factor III | ID-CMB305 | toll-like receptor 4 |
| | (thromboplastin, tissue | | cancer/testis antigen 1 |
| | factor) | | |
| MCS110 | colony stimulating factor 1 | imiquimod | toll-like receptor 7 |
| | (macrophage) | (intravesical), | |
| | | Telormedix | |
| ARRY-382 | colony stimulating factor 1 | NKTR-262 | toll-like receptor 7 |
| | (macrophage) | | toll-like receptor 8 |
| | colony stimulating factor 1 | | |
| | receptor | | |
| BLZ-945 | colony stimulating factor 1 | motolimod | toll-like receptor 8 |
| | receptor | | |
| AMG 820 | colony stimulating factor 1 | tilsotolimod | toll-like receptor 9 |
| | receptor | | |
| cabiralizumab | colony stimulating factor 1 | sacituzumab | topoisomerase (DNA) I |
| | receptor | govitecan | tumor-associated calcium |
| | | | signal transducer 2 |
| gemogenovatucel-T | colony stimulating factor 2 | HPV-16 E6 TCR, | transforming protein E6, |
| | (granulocyte-macrophage) | Bluebird Bio/Kite | human papilloma virus-16 |
| | | Pharma | |
| GVAX | colony stimulating factor 2 | VGX-3100 | transforming protein E6, |
| | (granulocyte-macrophage) | | human papilloma virus-16 |
| | | | transforming protein E7, |
| | | | human papilloma virus-16 |
| | | | E6 protein, human papilloma |
| | | | virus-18 |
| | | | E7 protein, human papilloma |
| | | | virus-18 |
| talimogene | colony stimulating factor 2 | MEDI0457 | transforming protein E6, |
| laherparepvec | (granulocyte-macrophage) | | human papilloma virus-16 |
| | | | transforming protein E7, |
| | | | human papilloma virus-16 |
| | | | E7 protein, human papilloma |
| | | | virus-18 |
| | | | E6 protein, human papilloma |
| | | | virus-18 |
| pexastimogene | colony stimulating factor 2 | TVGV-1 | transforming protein E7, |
| devacirepvec | (granulocyte-macrophage) | | human papilloma virus-16 |
| sargramostim | colony stimulating factor 2 | KITE-439 | transforming protein E7, |
| | receptor, alpha, low-affinity | | human papilloma virus-16 |
| | (granulocyte-macrophage) | | |
| SV-BR-1-GM | colony stimulating factor 2 | ADXS-DUAL | transforming protein E7, |
| cancer vaccine | receptor, alpha, low-affinity | | human papilloma virus-16 |
| | (granulocyte-macrophage) | | |
| pamrevlumab | connective tissue growth | axalimogene | transforming protein E7, |
| | factor | filolisbac | human papilloma virus-16 |
| ipilimumab | cytotoxic T-lymphocyte- | MVA-5T4 | trophoblast glycoprotein |
| | associated protein 4 | | |
| tremelimumab | cytotoxic T-lymphocyte- | oportuzumab | tumor-associated calcium |
| | associated protein 4 | monatox | signal transducer 2 |
| BMS-986249 | cytotoxic T-lymphocyte- | denosumab | tumour necrosis factor |
| | associated protein 4 | | (ligand) superfamily, member 11 |
| rovalpituzumab | delta-like 3 (Drosophila) | BION-1301 | tumour necrosis factor |
| tesirine | | | (ligand) superfamily, member 13 |
| ABT-165 | delta-like 4 (Drosophila) | belimumab | tumour necrosis factor |
| | vascular endothelial growth | | (ligand) superfamily, member 13b |
| | factor A | | |
| BHQ880 | dickkopf WNT signaling | INCAGN1876 | tumour necrosis factor |
| | pathway inhibitor 1 | | receptor superfamily, member 18 |
| DKN-01 | dickkopf WNT signaling | BMS-986156 | tumour necrosis factor |
| | pathway inhibitor 1 | | receptor superfamily, member 18 |
| Ad-REIC vaccine, | dickkopf WNT signaling | INCAGN1949 | tumour necrosis factor |
| Momotaro-Gene | pathway inhibitor 3 | | receptor superfamily, member 4 |
| AGS-16C3F | ectonucleotide | PF-04518600 | tumour necrosis factor |
| | pyrophosphatase/ | | receptor superfamily, member 4 |
| | phosphodiesterase 3 | | |
| carotuximab | endoglin | BMS-986178 | tumour necrosis factor |
| | | | receptor superfamily, member 4 |
| ifabotuzumab | EPH receptor A3 | brentuximab | tumour necrosis factor |
| | | vedotin | receptor superfamily, member 8 |
| CimaVax EGF | epidermal growth factor | urelumab | tumour necrosis factor |
| | (beta-urogastrone) | | receptor superfamily, member 9 |
| depatuxizumab | epidermal growth factor | utomilumab | tumour necrosis factor |
| mafodotin | receptor | | receptor superfamily, member 9 |
| RM-1929 | epidermal growth factor | VBI-1901 | UL83, cytomegalovirus |
| | receptor | | UL55, cytomegalovirus |
| AVID100 | epidermal growth factor | bevacizumab | vascular endothelial growth |
| | receptor | biosimilar, | factor A |
| | | Boehringer | |
| | | Ingelheim | |
| trastuzumab | epidermal growth factor | bevacizumab-awwb | vascular endothelial growth |
| biosimilar, | receptor | | factor A |
| Henlius | | | |
| cetuximab | epidermal growth factor | bevacizumab | vascular endothelial growth |
| | receptor | biosimilar, Pfizer | factor A |
| panitumumab | epidermal growth factor | bevacizumab | vascular endothelial growth |
| | receptor | biosimilar, | factor A |
| | | Oncobiologics | |
| necitumumab | epidermal growth factor | bevacizumab | vascular endothelial growth |
| | receptor | biosimilar, Henlius | factor A |
| | | Biopharmaceuticals | |
| nimotuzumab | epidermal growth factor | bevacizumab | vascular endothelial growth |
| | receptor | biosimilar, Fujifilm | factor A |
| | | Kyowa Kirin | |
| | | Biologics | |
| futuximab | epidermal growth factor | aflibercept | vascular endothelial growth |
| | receptor | | factor A |
| tomuzotuximab | epidermal growth factor | bevacizumab | vascular endothelial growth |
| | receptor | | factor A |
| doxorubicin, EDV | epidermal growth factor | pritumumab | vimentin |
| nanocells, | receptor | | |
| EnGeneIC | | | |
| pan-HER | epidermal growth factor | pexidartinib | v-kit Hardy-Zuckerman 4 |
| | receptor | | feline sarcoma viral oncogene |
| | erb-b2 receptor tyrosine | | homologue |
| | kinase 2 | | colony stimulating factor 1 |
| | erb-b2 receptor tyrosine | | receptor |
| | kinase 3 | | fms-related tyrosine kinase 3 |
| trastuzumab | erb-b2 receptor tyrosine | galinpepimut-S | Wilms tumour 1 |
| deruxtecan | kinase 2 | | |
| trastuzumab | erb-b2 receptor tyrosine | adegramotide/ | Wilms tumour 1 |
| emtansine | kinase 2 | nelatimotide | |
| (vic-)trastuzumab | erb-b2 receptor tyrosine | JTCR016 | Wilms tumour 1 |
| duocarmazine | kinase 2 | | |
| nelipepimut-S | erb-b2 receptor tyrosine | levamisole | Unknown |
| | kinase 2 | | |
| trastuzumab | erb-b2 receptor tyrosine | ladiratuzumab | Unknown |
| biosimilar, Merck | kinase 2 | vedotin | |
| & Co./Samsung | | | |
| Bioepis | | | |
| trastuzumab | erb-b2 receptor tyrosine | NSC-631570 | Unknown |
| biosimilar, | kinase 2 | | |
| Celltrion | | | |
| trastuzumab | erb-b2 receptor tyrosine | LN-145 | Unknown |
| biosimilar, Biocon | kinase 2 | | |
| trastuzumab | erb-b2 receptor tyrosine | INO-5401 | Unknown |
| biosimilar, | kinase 2 | | |
| Allergan/Amgen | | | |
| trastuzumab | erb-b2 receptor tyrosine | AN01, Anson | Unknown |
| biosimilar, Pfizer | kinase 2 | Pharma | |
| AU101, Aurora | erb-b2 receptor tyrosine | GALE-302 | Unknown |
| Biopharma | kinase 2 | | |
| AU105, Aurora | erb-b2 receptor tyrosine | MAGE-A3 TCR, | Unknown |
| BioPharma | kinase 2 | Adaptimmune | |
| AE37 | erb-b2 receptor tyrosine | BTH-1677 | Unknown |
| | kinase 2 | | |
| trastuzumab | erb-b2 receptor tyrosine | lentinan | Unknown |
| | kinase 2 | | |
| pertuzumab | erb-b2 receptor tyrosine | Polysaccharide-K | Unknown |
| | kinase 2 | | |
| margetuximab | erb-b2 receptor tyrosine | Tice BCG, Organon | Unknown |
| | kinase 2 | | |
| ADXS31-164 | erb-b2 receptor tyrosine | IGEM-F | Unknown |
| | kinase 2 | | |
| ETBX-021 | erb-b2 receptor tyrosine | PV-10, Provectus | Unknown |
| | kinase 2 | | |
| seribantumab | erb-b2 receptor tyrosine | vitespen | Unknown |
| | kinase 3 | | |
| patritumab | erb-b2 receptor tyrosine | mifamurtide | Unknown |
| | kinase 3 | | |
| CDX-3379 | erb-b2 receptor tyrosine | melanoma vaccine, | Unknown |
| | kinase 3 | GSK | |
| elgemtumab | erb-b2 receptor tyrosine | Bacille Calmette- | Unknown |
| | kinase 3 | Guerin vaccine, ID | |
| | | Biomedical | |
| moxetumomab | eukaryotic translation | seviprotimut-I | Unknown |
| pasudotox | elongation factor 2 | | |
| | CD22 molecule | | |
| denileukin diftitox | eukaryotic translation | in situ autologous | Unknown |
| | elongation factor 2 | cancer vaccine, | |
| | interleukin 2 receptor, alpha | Immunophotonics | |
| MDNA55 | eukaryotic translation | IMA901 | Unknown |
| | elongation factor 2 | | |
| | interleukin 4 receptor | | |
| bemarituzumab | fibroblast growth factor | adagloxad | Unknown |
| | receptor 2 | simolenin | |
| DCVax-prostate, | folate hydrolase (prostate- | PVX-410 | Unknown |
| Northwest | specific membrane antigen) 1 | | |
| Biotherapeutics | | | |
| 177Lu-J591 | folate hydrolase (prostate- | viagenpumatucel-L | Unknown |
| | specific membrane antigen) 1 | | |
| tuberculosis | folate hydrolase (prostate- | GALE-301 | Unknown |
| vaccine (Mw), | specific membrane antigen) 1 | | |
| Cadila; Cadi-05 | | | |
| mirvetuximab | folate receptor 1 (adult) | EP-302, EpiThany | Unknown |
| soravtansine | | | |
| TPIV200 | folate receptor 1 (adult) | BI 1361849 | Unknown |
| farletuzumab | folate receptor 1 (adult) | DPV-001 | Unknown |
| IGEM-FR | folate receptor 1 (adult) | Bacille Calmette- | Unknown |
| | | Guerin vaccine, | |
| | | Sanofi | |
| G17DT | gastrin | LAMP-Vax + pp65 | Unknown |
| | | DC, Immunomic | |
| | | Therapeutics | |
| codrituzumab | glypican 3 | NKG2D-CAR | Unknown |
| EP-100, | gonadotropin-releasing | BPX-501 | Unknown |
| EpiThany | hormone 1 (luteinizing- | | |
| | releasing hormone) | | |
| | luteinizing hormone/ | | |
| | choriogonadotropin receptor | | |
| naxitamab | growth differentiation factor 2 | NK-92 cells | Unknown |
| CDX-014 | hepatitis A virus cellular | LN-144 | Unknown |
| | receptor 1 | | |
| MBG453 | hepatitis A virus cellular | CLBS-23 | Unknown |
| | receptor 2 | | |
| histamine | histamine receptor H2 | DCVax-Direct, | Unknown |
| dihydrochloride | | Northwest | |
| | | Biotherapeutics | |
| entinostat | histone deacetylase 1 | melanoma vaccine, | Unknown |
| | | AVAX | |
| indoximod | indoleamine-pyrrole 2,3 | stapuldencel-T | Unknown |
| | dioxygenase | | |
| epacadostat | indoleamine-pyrrole 2,3 | dendritic cancer | Unknown |
| | dioxygenase | vaccine, DanDrit | |
| | | Biotech | |
| BMS-986205 | indoleamine-pyrrole 2,3 | DCVax-Brain | Unknown |
| | dioxygenase | brain cancer | |
| | | vaccine, Northwest | |
| | | Biotherapeutics | |
| JTX-2011 | inducible T-cell co- | tumor lysate | Unknown |
| | stimulator | particle-loaded | |
| | | dendritic cell | |
| | | vaccine, Perseus | |
| BMS-986226 | inducible T-cell co- | ERC1671 | Unknown |
| | stimulator | | |
| ADC W0101 | insulin-like growth factor 1 | BSK01 | Unknown |
| | receptor | TAPA pulsed DC | |
| | | vaccine | |
| ganitumab | insulin-like growth factor 1 | Oncoquest-CLL | Unknown |
| | receptor | vaccine | |
| istiratumab | insulin-like growth factor 1 | rocapuldencel-T | Unknown |
| | receptor | | |
| | erb-b2 receptor tyrosine | | |
| | kinase 3 | | |
| dusigitumab | insulin-like growth factor 1 | ATIR-101 | Unknown |
| | receptor | | |
| | insulin-like growth factor 2 | | |
| | receptor | | |
| EP-201, | insulin-like growth factor | TVI-Kidney-1 | Unknown |
| EpiThany | binding protein 2, 36 kDa | | |
| citoplurikin | interferon gamma receptor 1 | TVAX cancer | Unknown |
| | tumor necrosis factor | vaccine, TVAX | |
| | receptor superfamily, | Biomedical | |
| | member 1A | | |
| MABp1 | interleukin 1, alpha | atezolizumab, | Unknown |
| | | companion | |
| | | diagnostic | |
| pegilodecakin | interleukin 10 | tumour infiltrating | Unknown |
| | | lymphocytes, | |
| | | lovance | |
| | | Biotherapeutics-2 | |
| Ad-RTS-hIL-12 + | interleukin 12 receptor, beta 1 | MAGE A-10 TCR, | Unknown |
| veledimex | | Adaptimmune | |
| tavokinogene | interleukin 12 receptor, beta 1 | IMA101 | Unknown |
| telsaplasmid | interleukin 12 receptor, beta 2 | | |
| EGEN-001 | interleukin 12A (natural | algenpantucel-L | Unknown |
| | killer cell stimulatory factor | | |
| | 1, cytotoxic lymphocyte | | |
| | maturation factor 1, p35) | | |
| | interleukin 12B (natural | | |
| | killer cell stimulatory factor | | |
| | 2, cytotoxic lymphocyte | | |
| | maturation factor 2, p40) | | |
| SL-701 | interleukin 13 receptor, alpha 2 | Tumor Necrosis | Unknown |
| | EPH receptor A2 | Therapy, Peregrine | |
| | baculoviral IAP repeat | | |
| | containing 5 | | |
| ALT-803 | interleukin 15 receptor, alpha | imiquimod | Unknown |
| Multikine, Cel-Sci | interleukin 2 receptor, alpha | LOAd703 | Unknown |
| ALT-801 | interleukin 2 receptor, alpha | CG0070 | Unknown |
| high-affinity | interleukin 2 receptor, alpha | dinutuximab | Unknown |
| Natural Killer | | | |
| (haNK) cells, | | | |
| NantKwest | | | |
| interleukin-2, | interleukin 2 receptor, alpha | bavituximab | Unknown |
| Roche | | | |
| aldesleukin | interleukin 2 receptor, alpha | ensituximab | Unknown |
| NKTR-214 | interleukin 2 receptor, beta | pidilizumab | Unknown |
| talacotuzumab | interleukin 3 receptor, alpha | BMS-986218 | Unknown |
| | (low affinity) | | |
| SL-401 | interleukin 3 receptor, alpha | BMS-986012 | Unknown |
| | (low affinity) | | |
| siltuximab | interleukin 6 (interferon, | ADXS31-142 | Unknown |
| | beta 2) | | |
| HuMax-IL8 | interleukin 8 | GI-6301 | Unknown |
| PSA/IL-2/GM- | kallikrein-related peptidase 3 | GI-4000 | Unknown |
| CSF | | | |
| rilimogene | kallikrein-related peptidase 3 | JNJ-64041757 | Unknown |
| galvacirepvec | CD80 molecule | | |
| | intercellular adhesion | | |
| | molecule 1 | | |
| | CD58 molecule | | |
| monalizumab | killer cell lectin-like receptor | HPV vaccine | Unknown |
| | subfamily C, member 1 | (Cervarix), GSK | |
| ramucirumab | kinase insert domain receptor | HPV vaccine | Unknown |
| | | (Gardasil), CSL | |
| ubenimex | leucotriene A4 hydrolase | Sym015 | Unknown |
| | leucotriene B4 receptor | | |
| IMP321 | lymphocyte-activation gene 3 | diphenylcyclopropenone | Unknown |
| LAG525 | lymphocyte-activation gene 3 | ISA101 | Unknown |
| relatlimab | lymphocyte-activation gene 3 | | |

TABLE 3
Sequences

| Name | | SEQ | |
|---|---|---|---|
| (Target, if | | ID | |
| applicable) | Region | NO | Sequence |
| JTX-2011 | Heavy | 1 | EVQLVESGGGLVQPGGSLRLSCAASGFTFSDYWMDWVRQAPGKGLVWVSNI |
| (ICOS) | Chain | | DEDGSITEYSPFVKGRFTISRDNAKNTLYLQMNSLRAEDTAVYYCTRWGRF |
| | | | GFDSWGQGTLVTVSSASTKGPSVFPLAPSSKSTSGGTAALGCLVKDYFPEP |
| | | | VTVSWNSGALTSGVHTFPAVLQSSGLYSLSSVVTVPSSSLGTQTYICNVNH |
| | | | KPSNTKVDKKVEPKSCDKTHTCPPCPAPELLGGPSVFLFPPKPKDTLMISR |
| | | | TPEVTCVVVDVSHEDPEVKFNWYVDGVEVHNAKTKPREEQYNSTYRVVSVL |
| | | | TVLHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPG |
| JTX-2011 | Light | 2 | DIVMTQSPDSLAVSLGERATINCKSSQSLLSGSFNYLTWYQQKPGQ |
| (ICOS) | Chain | | PPKLLIFYASTRHTGVPDRFSGSGSGTDFTLTISSLQAEDVAVYYC |
| | | | HHHYNAPPTFGPGTKVDIKRTVAAPSVFIFPPSDEQLKSGTASVVC |
| | | | LLNNFYPREAKVQWKVDNALQSGNSQESVTEQDSKDSTYSLSSTLT |
| | | | LSKADYEKHKVYACEVTHQGLSSPVTKSFNRGEC |
| JTX-2011 | Heavy | 3 | EVQLVESGGGLVQPGGSLRLSCAASGFTFSDYWMDWVRQAPGKGLVWVSNI |
| (ICOS) | Chain | | DEDGSITEYSPFVKGRFTISRDNAKNTLYLQMNSLRAEDTAVYYCTRWGRF |
| | Variable | | GFDSWGQGTLVTVSS |
| JTX-2011 | Light | 4 | DIVMTQSPDSLAVSLGERATINCKSSQSLLSGSFNYLTWYQQKPGQPPKLL |
| (ICOS) | Chain | | IFYASTRHTGVPDRFSGSGSGTDFTLTISSLQAEDVAVYYCHHHYNAPPTF |
| | Variable | | GPGTKVDIK |
| JTX-2011 | HCDR1 | 5 | GFTFSDYWMD |
| (ICOS) | | | |
| JTX-2011 | HCDR2 | 6 | NIDEDGSITEYSPFVK |
| (ICOS) | | | |
| JTX-2011 | HCDR3 | 7 | WGRFGFDS |
| (ICOS) | | | |
| JTX-2011 | LCDR1 | 8 | KSSQSLLSGSFNYLT |
| (ICOS) | | | |
| JTX-2011 | LCDR2 | 9 | YASTRHT |
| (ICOS) | | | |
| JTX-2011 | LCDR3 | 10 | HHHYNAPPT |
| (ICOS) | | | |
| Human ICOS | | 11 | MKSGLWYFFLFCLRIKVLTGEINGSANYEMFIFHNGGVQILCKYPDIVQQF |
| precursor | | | KMQLLKGGQILCDLTKTKGSGNTVSIKSLKFCHSQLSNNSVSFFLYNLDHS |
| with signal | | | HANYYFCNLSIFDPPPFKVTLTGGYLHIYESQLCCQLKFWLPIGCAAFVVV |
| sequence | | | CILGCILICWLTKKKYSSSVHDPNGEYMFMRAVNTAKKSRLTDVTL |
| | | | (Intra-cellar Region is under-lined) |
| Human ICOS, | | 12 | EINGSANYEMFIFHNGGVQILCKYPDIVQQFKMQLLKGGQILCDLTKTKGS |
| mature | | | GNTVSIKSLKFCHSQLSNNSVSFFLYNLDHSHANYYFCNLSIFDPPPFKVT |
| | | | LTGGYLHIYESQLCCQLKFWLPIGCAAFVVVCILGCILICWLTKKKYSSSV |
| | | | HDPNGEYMFMRAVNTAKKSRLTDVTL |
| | | | (Intra-cella Region is under-lined) |
| Mouse (Mus | | 13 | MKPYFCRVFVFCFLIRLLTGEINGSADHRMFSFHNGGVQISCKYPE |
| musculus) | | | TVQQLKMRLFREREVLCELTKTKGSGNAVSIKNPMLCLYHLSNNSV |
| ICOS | | | SFFLNNPDSSQGSYYFCSLSIFDPPPFQ |
| precursor | | | ERNLSGGYLHIYESQLCCQLKLWLPVGCAAFVVVLLFGCILIIWFS |
| | | | KKKYGSSVHDPNSEYMFMAAVNTNKKSRLAGVTS |
| Mouse (Mus | | 14 | EINGSADHRMFSFHNGGVQISCKYPETVQQLKMRLFREREVLCELT |
| musculus) | | | KTKGSGNAVSIKNPMLCLYHLSNNSVSFFLNNPDSSQGSYYFCSLS |
| ICOS, mature | | | IFDPPPFQERNLSGGYLHIYESQLCCQLKLWLPVGCAAFVVVLLFG |
| | | | CILIIWFSKKKYGSSVHDPNSEYMFMAAVNTNKKSRLAGVTS |
| Rat (Rattus | | 15 | MKPYFSCVFVFCFLIKLLTGELNDLANHRMFSFHDGGVQISCNYPE |
| norvegicus) | | | TVQQLKMQLFKDREVLCDLTKTKGSGNTVSIKNPMSCPYQLSNNSV |
| ICOS | | | SFFLDNADSSQGSYFLCSLSIFDPPPFQEKNLSGGYLLIYESQLCC |
| precursor | | | QLKLWLPVGCAAFVAALLFGCIFIVWFAKKKYRSSVHDPNSEYMFM |
| | | | AAVNTNKKSRLAGMTS |
| Rat (Rattus | | 16 | ELNDLANHRMFSFHDGGVQISCNYPETVQQLKMQLFKDREVLCDLTKTKGS |
| norvegicus) | | | GNTVSIKNPMSCPYQLSNNSVSFFLDNADSSQGSYFLCSLSIFDPPPFQEK |
| ICOS, mature | | | NLSGGYLLIYESQLCCQLKLWLPVGCAAFVAALLFGCIFIVWFAKKKYRSS |
| | | | VHDPNSEYMFMAAVNTNKKSRLAGMTS |
| Cynomolgus | | 17 | MKSGLWYFFLFCLHMKVLTGEINGSANYEMFIFHNGGVQILCKYPDIVQQF |
| monkey | | | KMQLLKGGQILCDLTKTKGSGNKVSIKSLKFCHSQLSNNSVSFFLYNLDRS |
| (Macaca | | | HANYYFCNLSIFDPPPFKVTLTGGYLHIYESQLCCQLKFWLPIGCATFVVV |
| fascicularis) | | | CIFGCILICWLTKKKYSSTVHDPNGEYMFMRAVNTAKKSRLTGTTP |
| ICOS, | | | |
| precursor | | | |
| Cynomolgus | | 18 | EINGSANYEMFIFHNGGVQILCKYPDIVQQFKMQLLKGGQILCDLT |
| monkey | | | KTKGSGNKVSIKSLKFCHSQLSNNSVSFFLYNLDRSHANYYFCNLS |
| (Macaca | | | IFDPPPFKVTLTGGYLHIYESQLCCQLKFWLPIGCATFVVVCIFGC |
| fascicularis) | | | ILICWLTKKKYSSTVHDPNGEYMFMRAVNTAKKSRLTGTTP |
| ICOS, mature | | | |
| JNC-1 (PD-1) | Heavy | 19 | QVQLVQSGAEVKKPGASVKVSCKASGYTFPSYYMHWVRQAPGQGLEWMGII |
| | Chain | | NPEGGSTAYAQKFQGRVTMTRDTSTSTVYMELSSLRSEDTAVYYCARGGTY |
| | Variable | | YDYTYWGQGTLVTVSS |
| | Region | | |
| JNC-1 (PD-1) | HCDR1 | 20 | YTFPSYYMH |
| JNC-1 (PD-1) | HCDR2 | 21 | IINPEGGSTAYAQKFQG |
| JNC-1 (PD-1) | HCDR3 | 22 | ARGGTYYDYTY |
| JNC-1 (PD-1) | Light | 23 | DIQMTQSPSTLSASVGDRVTITCRASQSISSWLAWYQQKPGKAPKLLIYEA |
| | Chain | | SSLESGVPSRFSGSGSGTEFTLTISSLQPDDFATYYCQQYNSFPPTFGGGT |
| | Variable | | KVEIK |
| | Region | | |
| JNC-1 (PD-1) | LCDR1 | 24 | RASQSISSWLA |
| JNC-1 (PD-1) | LCDR2 | 25 | EASSLES |
| JNC-1 (PD-1) | LCDR3 | 26 | QQYNSFPPT |
| 2M13 (ICOS | Heavy | 27 | EVQLQQSGAELVRPGAVVKLSCKASGFDIKDYYMHWVQQRPEQGLEWIGWI |
| intra- | Chain | | DPENGNAVYDPQFQGKASITADTSSNTAYLQLSSLTSEDTAVYYCASDYYG |
| cellular) | Variable | | SKGYLDVWGAGTTVTVSS |
| | Region | | |
| 2M13 (ICOS | HCDR1 | 28 | DYYMH |
| intra- | | | |
| cellular) | | | |
| 2M13 (ICOS | HCDR2 | 29 | WIDPENGNAVYDPQFQG |
| intra- | | | |
| cellular) | | | |
| 2M13 (ICOS | HCDR3 | 30 | |
| intra- | | | DYYGSKGYLDV |
| cellular) | | | |
| 2M13 (ICOS | Light | 31 | QIVLTQSPTIMSASPGEKVTITCSASSSVSYMHWFQQKPGTSPKLWIYSTS |
| intra- | Chain | | NLASGVPARFGGSRSGTSYSLTISRMEAEDAATYYCQQRSSYPFTFGSGTK |
| cellular) | Variable | | LEIK |
| | Region | | |
| 2M13 (ICOS | LCDR1 | 32 | SASSSVSYMH |
| intra- | | | |
| cellular) | | | |
| 2M13 (ICOS | LCDR2 | 33 | STSNLAS |
| intra- | | | |
| cellular) | | | |
| 2M13 (ICOS | LCDR3 | 34 | QQRSSYPFT |
| intra- | | | |
| cellular) | | | |
| 2M19 (ICOS | Heavy | 35 | EVQLQQSGAELVRSGASVKLSCTTSAFNIIDYYMHWVIQRPEQGLEWIAWI |
| intra- | Chain | | DPENGDPEYAPKFQDKATMTTDTSSNTAYLQLSSLTSEDTAVYYCTAWRGF |
| cellular) | Variable | | AYWGQGTLVTVSA |
| | Region | | |
| 2M19 (ICOS | HCDR1 | 36 | DYYMH |
| intra- | | | |
| cellular) | | | |
| 2M19 (ICOS | HCDR2 | 37 | WIDPENGDPEYAPKFQD |
| intra- | | | |
| cellular) | | | |
| 2M19 (ICOS | HCDR3 | 38 | WRGFAY |
| intra- | | | |
| cellular) | | | |
| 2M19 (ICOS | Light | 39 | DVVMTQTPLSLPVSLGDQASISCRSSQSLVHSNGNTYLHWYLQKPGQSPKL |
| intra- | Chain | | LIYKVSNRFSGVPDRFSGSGSGTDFTLKISRVEAEDLGVYFCSQSIHVPPT |
| cellular) | Variable | | FGGGTKLEIK |
| | Region | | |
| 2M19 (ICOS | LCDR1 | 40 | |
| intra- | | | RSSQSLVHSNGNTYLH |
| cellular) | | | |
| 2M19 (ICOS | LCDR2 | 41 | KVSNRFS |
| intra- | | | |
| cellular) | | | |
| 2M19 (ICOS | LCDR3 | 42 | SQSIHVPPT |
| intra- | | | |
| cellular) | | | |

What is claimed is:

1. A method of generating a population ICOShi, CD4+ T cells, said method comprising isolating immune cells from a patient, contacting said immune cells with (i) one or more antigens, and (ii) an ICOS agonist, wherein said contacting results in the generation of a population of ICOShi CD4+ T-cells.

2. The method of claim 1, wherein said contacting with one or more antigen precedes contacting with said ICOS agonist.

3. The method of claim 1 or 2, wherein said isolated immune cells are isolated peripheral blood mononuclear cells.

4. The method of any one of claims 1-3, wherein said contacting of said immune cells with said antigen further comprises contacting said immune cells with one or more Th1 skewing compounds and anti-CD28.

5. The method of claim 4, wherein said one or more Th1 skewing compounds are selected from the group consisting of IL-12, IL-2, anti-IL-4, IL-15, IL-18, and IL-21 (e.g., said one or more Th1 skewing compounds comprise IL-12, IL-2, and anti-IL-4).

6. The method of any one of claims 1-5, wherein said ICOS agonist is an anti-ICOS antibody agonist, e.g., wherein the anti-ICOS antibody agonist comprises (a) a heavy chain comprising the amino acid sequence of SEQ ID NO: 1 and/or (b) a light chain comprising the amino acid sequence of SEQ ID NO: 2.

7. The method of claim 6, wherein said anti-ICOS antibody agonist is selected from the group consisting of JTX-2011, BMS-986226, and GSK3359609.

8. The method of any one of claims 1-7, wherein said method comprises contacting said immune cells with two or more antigens (e.g., 10 or more antigens, 100 or more antigens, 1000 or more antigens, or 10,000 or more antigens).

9. The method of any one of claim 1-8, wherein one or more of said antigens is an antigen associated with an infectious disease.

10. The method of claim 9, wherein said infectious disease is selected from HIV, tuberculosis, Epstein Barr virus, herpes simplex virus, malaria, and human papilloma virus.

11. The method of any one of claims 1-8, wherein one or more of said antigens is a cancer-associated antigen.

12. The method of claim 11, wherein one or more of said cancer-antigens results from a mutation in a gene selected from the group consisting of: TP53, PIK3CA, TTN, KRAS, APC, MUC16, KMT2D, KMT2C, ARID1A, PTEN, BRAF, IDH1, NRAS, AKT1, and EGFR.

13. The method of claim 11, wherein one or more of said cancer-antigens results from a mutation selected from the group consisting of: TP53 (R273, R248, R175, R213, G245, R282, Y220, and H179), PIK3CA (H1047, E545, and E542), TTN, KRAS (G12, G13, and 061), APC, MUC16, KMT2D, KMT2C, ARID1A, PTEN (R130), BRAF (V600), IDH1 (R132), NRAS (Q61), AKT1 (E17), and EGFR (745-759 in-frame indel).

14. The method of claim 11, wherein one or more of said cancer-antigen results from a mutation identified in Chang et al. Cancer discovery 8.2 (2018): 174-183 or Chang et al. Nature biotechnology 34.2 (2016): 155.

15. The method of any one of claims 1-14, wherein contacting said immune cells with an ICOS agonist further comprises contacting said immune cells with an additional immunomodulatory agent.

16. The method of claim 15, wherein said additional immunomodulatory agent is selected from the group consisting of an anti-CD3 antibody, an anti-PD-1 antagonist antibody, an anti-PD-L1 antagonist antibody, an anti-CTLA4 antibody.

17. The method of claim 16, wherein the anti-PD-1 or anti-PD-Li antagonist antibody is selected from the group consisting of avelumab, atezolizumab, CX-072, pembrolizumab, nivolumab, cemiplimab, spartalizumab, tislelizumab, JNJ-63723283, genolimzumab, AMP-514, AGEN2034, durvalumab, and JNC-1.

18. The method of claim 16, wherein the anti-CTLA4 antibody is selected from the group consisting of ipilimumab, tremelimumab, and BMS-986249.

19. The method of any one of claims 1-18, wherein said method further comprises isolating said ICOShi, CD4+ T cells.

20. The method of any one of claims 1-18, wherein said method further comprises culturing said ICOShi, CD4+ T cells.

21. The method of claim 19, wherein said method further comprises culturing said isolated ICOShi, CD4+ T cells.

22. The method of claim 20 or 21, wherein said culturing comprises initial culture conditions suitable for expanding said population of ICOShi, CD4+ T cells.

23. The method of claim 22, wherein said initial conditions suitable for expanding said population ICOShi, CD4+ T cells comprise contacting said suspension of CD4+ T cells with a CD3 agonist.

24. The method of claim 23, wherein said CD3 agonist is an anti-CD3 antibody (e.g., OKT3).

25. The method of any one of claims 22-24, wherein said initial conditions suitable for expanding said population of ICOShi, CD4+ T cells comprise contacting the cells with one or more of an anti-PD-1 antibody antagonist, an anti-CTLA-4 antibody, and an ICOS agonist.

26. The method of any one of claims 22-25, wherein said initial conditions suitable for expanding said population of ICOShi, CD4+ T cells comprise contacting the cells with one or more compounds (e.g., two or more, or all three) selected from the group consisting of IL-2, IL-12 and anti-IL-4.

27. The method of any one of claims 22-26, wherein said initial conditions suitable for expanding said population of ICOShi, CD4+ T cells comprise contacting the cells with an anti-CD28 antibody agonist.

28. The method of any one of claims 22-26, wherein said CD3 agonist and anti-CD28 agonist are present in a tetrameric antibody complex.

29. The method of any one of claims 22-27, wherein said suspension of ICOShi, CD4+ T cells are incubated under said initial culture conditions for a period between one and five days (e.g., approximately 1, 2, 3, 4, or five days).

30. The method of claim 29, further comprising incubating said suspension of ICOShi, CD4+ T cells under a second culture condition suitable for expanding said population of ICOShi, CD4+ T cells.

31. The method of claim 30, wherein said cells are washed prior to the application of said second culture condition.

32. The method of claim 30 or 31, wherein said second culture condition comprises contacting the cells with one or more of an anti-PD-1 antibody antagonist, an anti-CTLA-4 antibody, and an ICOS agonist.

33. The method of any one of claims 30-32, wherein said second culture condition comprises contacting the cells with one or more compounds (e.g., two or more, or all three) selected from the group consisting of IL-2, IL-12 and anti-IL-4.

34. The method of any one of claims 30-33, wherein said second culture condition comprises contacting the cells with an anti-CD28 antibody agonist.

35. The method of any one of claims 30-34, wherein said second culture conditions does not comprise contacting the cells with a CD3 agonist and/or CD28 agonist.

36. The method of any one of claims 30-35, wherein said second culture condition is maintained for between 1 and 5 days (e.g., for 1, 2, 3, 4, or 5 days).

37. A suspension of cells generated by any one of the methods of claims 30-36.

38. A pharmaceutical composition comprising a suspension of cells of claim 37.

39. The method of any one of claims 21-36, wherein said method further comprises isolating said ICOShi, CD4+ T cells after said culturing.

40. The method of any one of claim 20 or 38, wherein said isolating comprises contacting said cells with an anti-ICOS antibody that does not compete with binding to ICOS with said ICOS agonist.

41. The method of claim 20 or 38, wherein said isolating comprises contacting said cells with an antibody specific for said ICOS agonist (e.g., an anti-human-IgG1 antibody), or with protein A/G beads.

42. The method of any one of claims 1-36, wherein said method further comprises administering said ICOShi CD4+ T-cells (e.g., said isolated ICOShi CD4+ T-cells) to said patient, thereby treating said patient for a disease associated with said one or more antigens.

43. The method of claim 41, wherein said one or more antigens is associated with cancer and said disease is cancer.

44. The method of claim 42, wherein said cancer is selected from gastric cancer, breast cancer, which optionally is triple negative breast cancer (TNBC), non-small cell lung cancer (NSCLC), melanoma, renal cell carcinoma (RCC), bladder cancer, endometrial cancer, diffuse large B-cell lymphoma (DLBCL), Hodgkin's lymphoma, ovarian cancer, and head and neck squamous cell cancer (HNSCC).

45. The method of claim 41, wherein said one or more antigens is associated with an infectious disease and said disease is an infectious disease.

46. The method of any one of claims 1-44, wherein, prior to said isolation of said immune cells from said patient, said patient is treated with an anti-cancer treatment.

47. The method of claim 45, wherein said anti-cancer treatment is selected from the group consisting of an agonist to TLR9, TLR2/4, TLR7/8, and/or TLR7, radiation, chemotherapies, tyrosine kinase inhibitors, epigenetic modifiers (e.g., HDAC inhibitors and demethylating agents), IMIDs (immuno-modulatory drugs, e.g., lenalidomide), cytokines (e.g., interferon gamma), anti-CTLA-4 antibodies, oncolytic viruses, vaccines, CD40 agonists, and ICOS agonists.

48. The method of claim 46, wherein said anti-CTLA-4 antibody is selected from the group consisting of ipilimumab, tremelimumab, and BMS-986249.

49. The method of claim 46, wherein said anti-cancer treatment ICOS agonist is an anti-ICOS antibody agonist, e.g., wherein the anti-ICOS antibody agonist comprises (a) a heavy chain comprising the amino acid sequence of SEQ ID NO: 1 and/or (b) a light chain comprising the amino acid sequence of SEQ ID NO: 2.

50. The method of claim 46, wherein said anti-cancer treatment anti-ICOS antibody agonist is selected from the group consisting of JTX-2011, BMS-986226, and GSK3359609.

51. A population of ICOShi, CD4+ T-cells generated by the method of any one of claims 1-37.

52. The method of any one of claims 1-51, wherein said patient is determined to have a disease associated with said one or more antigens prior to said contacting of said immune cells with one or more antigens.

53. A method of treating a subject having cancer or infectious disease, the method comprising administering to the subject ICOShi CD4+ T-cells, wherein said cells are optionally obtained using a method of any one of claims 1-36, 39-50, or 52.

54. A method of generating a population of ICOShi CD4+ T cells, said method comprising contacting immune cells isolated from a patient with (i) one or more antigens, and (ii) an ICOS agonist, wherein said contacting results in the generation of a population of ICOShi CD4+ T-cells.

55. Use of one or more antigens and an ICOS agonist for the generation of a population of ICOShi CD4+ T cells from immune cells isolated from a patient.

56. Use of ICOShi CD4+ T-cells in the treatment of a subject having cancer or an infectious disease, wherein said cells are optionally obtained using a method of any one of claims 1-36, 39-50, 52, or 54.


SEQUENCE LISTING (42 sequences)

SEQ ID NO: 1 - protein, 446 aa, Artificial Sequence
  Synthetic Construct
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 218 aa, Artificial Sequence
  Synthetic Construct
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 117 aa, Artificial Sequence
  Synthetic Construct
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 4 - protein, 111 aa, Artificial Sequence
  Synthetic Construct
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 5 - protein, 10 aa, Artificial Sequence
  Synthetic Construct
GFTFSDYWMD

SEQ ID NO: 6 - protein, 16 aa, Artificial Sequence
  Synthetic Construct
NIDEDGSITEYSPFVK

SEQ ID NO: 7 - protein, 8 aa, Artificial Sequence
  Synthetic Construct
WGRFGFDS

SEQ ID NO: 8 - protein, 15 aa, Artificial Sequence
  Synthetic Construct
KSSQSLLSGSFNYLT

SEQ ID NO: 9 - protein, 7 aa, Artificial Sequence
  Synthetic Construct
YASTRHT

SEQ ID NO: 10 - protein, 9 aa, Artificial Sequence
  Synthetic Construct
HHHYNAPPT

SEQ ID NO: 11 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 12 - protein, 179 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 13 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 14 - protein, 180 aa, Mus musculus
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 15 - protein, 200 aa, Rattus norvegicus
MKPYFSCVFVFCFLIKLLTGELNDLANHRMFSFHDGGVQISCNYPETVQQLKMQLFKDREVLCDLTKTKGSGNTVSIKNPMSCPYQLSNNSVSFFLDNADSSQGSYFLCSLSIFDPPPFQEKNLSGGYLLIYESQLCCQLKLWLPVGCAAFVAALLFGCIFIVWFAKKKYRSSVHDPNSEYMFMAAVNTNKKSRLAGMTS

SEQ ID NO: 16 - protein, 180 aa, Rattus norvegicus
ELNDLANHRMFSFHDGGVQISCNYPETVQQLKMQLFKDREVLCDLTKTKGSGNTVSIKNPMSCPYQLSNNSVSFFLDNADSSQGSYFLCSLSIFDPPPFQEKNLSGGYLLIYESQLCCQLKLWLPVGCAAFVAALLFGCIFIVWFAKKKYRSSVHDPNSEYMFMAAVNTNKKSRLAGMTS

SEQ ID NO: 17 - protein, 199 aa, Macaca fascicularis
MKSGLWYFFLFCLHMKVLTGEINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 18 - protein, 179 aa, Macaca fascicularis
EINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 19 - protein, 118 aa, Artificial Sequence
  Synthetic Construct
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSS

SEQ ID NO: 20 - protein, 9 aa, Artificial Sequence
  Synthetic Construct
YTFPSYYMH

SEQ ID NO: 21 - protein, 17 aa, Artificial Sequence
  Synthetic Construct
IINPEGGSTAYAQKFQG

SEQ ID NO: 22 - protein, 11 aa, Artificial Sequence
  Synthetic Construct
ARGGTYYDYTY

SEQ ID NO: 23 - protein, 107 aa, Artificial Sequence
  Synthetic Construct
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIK

SEQ ID NO: 24 - protein, 11 aa, Artificial Sequence
  Synthetic Construct
RASQSISSWLA

SEQ ID NO: 25 - protein, 7 aa, Artificial Sequence
  Synthetic Construct
EASSLES

SEQ ID NO: 26 - protein, 9 aa, Artificial Sequence
  Synthetic Construct
QQYNSFPPT

SEQ ID NO: 27 - protein, 120 aa, Artificial Sequence
  Synthetic Construct
EVQLQQSGAELVRPGAVVKLSCKASGFDIKDYYMHWVQQRPEQGLEWIGWIDPENGNAVYDPQFQGKASITADTSSNTAYLQLSSLTSEDTAVYYCASDYYGSKGYLDVWGAGTTVTVSS

SEQ ID NO: 28 - protein, 5 aa, Artificial Sequence
  Synthetic Construct
DYYMH

SEQ ID NO: 29 - protein, 17 aa, Artificial Sequence
  Synthetic Construct
WIDPENGNAVYDPQFQG

SEQ ID NO: 30 - protein, 11 aa, Artificial Sequence
  Synthetic Construct
DYYGSKGYLDV

SEQ ID NO: 31 - protein, 106 aa, Artificial Sequence
  Synthetic Construct
QIVLTQSPTIMSASPGEKVTITCSASSSVSYMHWFQQKPGTSPKLWIYSTSNLASGVPARFGGSRSGTSYSLTISRMEAEDAATYYCQQRSSYPFTFGSGTKLEIK

SEQ ID NO: 32 - protein, 10 aa, Artificial Sequence
  Synthetic Construct
SASSSVSYMH

SEQ ID NO: 33 - protein, 7 aa, Artificial Sequence
  Synthetic Construct
STSNLAS

SEQ ID NO: 34 - protein, 9 aa, Artificial Sequence
  Synthetic Construct
QQRSSYPFT

SEQ ID NO: 35 - protein, 115 aa, Artificial Sequence
  Synthetic Construct
EVQLQQSGAELVRSGASVKLSCTTSAFNIIDYYMHWVIQRPEQGLEWIAWIDPENGDPEYAPKFQDKATMTTDTSSNTAYLQLSSLTSEDTAVYYCTAWRGFAYWGQGTLVTVSA

SEQ ID NO: 36 - protein, 5 aa, Artificial Sequence
  Synthetic Construct
DYYMH

SEQ ID NO: 37 - protein, 17 aa, Artificial Sequence
  Synthetic Construct
WIDPENGDPEYAPKFQD

SEQ ID NO: 38 - protein, 6 aa, Artificial Sequence
  Synthetic Construct
WRGFAY

SEQ ID NO: 39 - protein, 112 aa, Artificial Sequence
  Synthetic Construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSIHVPPTFGGGTKLEIK

SEQ ID NO: 40 - protein, 16 aa, Artificial Sequence
  Synthetic Construct
RSSQSLVHSNGNTYLH

SEQ ID NO: 41 - protein, 7 aa, Artificial Sequence
  Synthetic Construct
KVSNRFS

SEQ ID NO: 42 - protein, 9 aa, Artificial Sequence
  Synthetic Construct
SQSIHVPPT